SEQUENCE LISTING (5 sequences)

SEQ ID NO: 1 - protein, 61 aa, Hemachatus haemachatus
  UNSURE (1)..(61)
LKCKNKLVPFLSKTCPEGKNLCYKMTMLKMPKIPIKRGCTDACPKSSLLVKVVCCNKDKCN

SEQ ID NO: 2 - protein, 61 aa, Hemachatus haemachatus
  UNSURE (1)..(61)
LKCKNKVVPFLKCKNKVVPFLCYKMTLKKVPKIPIKRGCTDACPKSSLLVNVMCCKTDKCN

SEQ ID NO: 3 - protein, 42 aa, Hemachatus haemachatus
  UNSURE (1)..(42)
  UNSURE (15)..(16): Xaa is an amino acid residue
LKCKNKLVPFLSKTXXCPEGKNXXLCYKMTXLKKVTPKIKRG

SEQ ID NO: 4 - protein, 42 aa, Hemachatus haemachatus
  UNSURE (1)..(42)
  UNSURE (12)..(12): Xaa is an amino acid residue
LKCKNKVVPFLXKTXXCKNKVVPFLCYKMTXLKKVTPKIKRG

SEQ ID NO: 5 - protein, 57 aa, Hemachatus haemachatus
  UNSURE (1)..(57)
LKCKNKVVPFLKCKNKVVPFLCYKMTLKKVPKIPIKRGCTDACPKSSLLVNVMCCKT

(19) United States
(12) Patent Application Publication
(10) Pub. No.: US 2009/0180995 A1
(43) Pub. Date: Jul. 16, 2009

(54) NOVEL ANTICOAGULANT POLYPEPTIDES AND COMPLEX

(75) Inventors: Ramachandra Manjunatha Kini, Singapore (SG); Yajnavalka Banerjee, Mumbai (IN)

Correspondence Address: PATENT CORRESPONDENCE;ARNALL GOLDEN GREGORY LLP, 171 17TH STREET NW, SUITE 2100, ATLANTA, GA, 30363, US

(73) Assignee: National University of Singapore

(21) Appl. No.: 11/997,733

(22) PCT Filed: Aug. 4, 2006
(86) PCT No.: PCT/SG2006/000222

§ 371 Date: Feb. 12, 2009

Related U.S. Application Data
(60) Provisional application No. 60/706,250, filed on Aug. 5, 2005.

(51) Int. Cl. A61K 38/17; C07K 14/435; C07H 21/04; C12N 15/63; C12N 5/10; C12P 21/02; C07K 1/00; C07K 1/04; C07K 1/14; C07K 16/00; G01N 33/53; A61K 31/7088; A61P 7/02; A61K 35/12

(52) U.S. Cl. 424/93.7; 530/324; 536/23.5; 536/24.33; 536/24.31; 435/320.1; 435/325; 435/69.1; 530/333; 530/334; 530/344; 530/389.1; 435/7.1; 514/12; 514/44

(57) ABSTRACT

This invention is in the field of snake venom and the invention provides two novel snake polypeptides and nucleic acids encoding the same. Also provided are various uses methods and compositions based on the discovery of the novel snake polypeptides and their ability to synergistically inhibit coagulation.

All documents cited herein are incorporated by reference in their entirety.

### TECHNICAL FIELD

This invention is in the field of snake venom and the invention provides two novel snake polypeptides and nucleic acids encoding the same. Also provided are various uses, methods and compositions based on the discovery of the novel snake polypeptides and their ability to inhibit blood coagulation.

### BACKGROUND ART

Blood coagulation is an innate response to vascular injury that results from a series of amplified reactions, in which specific zymogens of serine proteases circulating in the plasma are sequentially activated by limited proteolysis leading to the formation of blood clot, thereby preventing the loss of blood (1-3). It is initiated through the extrinsic pathway (4). Membrane bound tissue factor (TF), which is exposed as a result of vascular injury, interacts with factor VIIa (FVIIa), which is preexistent in the plasma (at 1%-2% of the total factor VII) (5, 6), and forms the extrinsic tenase complex. This complex activates factor X (FX) to factor Xa (FXa). In association with its cofactor factor Va, FXa performs a proteolytic cleavage of prothrombin to thrombin. Thrombin cleaves fibrinogen to fibrin, promoting formation of a fibrin clot and activates platelets for inclusion in the clot. The TF-FVIIa complex can also activate factor IX (FIX) to factor IXa (FIXa), thus helping in the propagation of the coagulation cascade through the intrinsic pathway. The coagulation cascade is under tight regulation. Any imbalance in its regulation could lead to either unclottable blood that results in excessive bleeding during injuries or unwanted clot formation resulting in death and debilitation due to vascular occlusion with the consequence of myocardial infarction, stroke, pulmonary embolism, or venous thrombosis (7). Therefore, there is an urgent need for the prophylaxis and treatment of thromboembolic disorders.

Anticoagulants are pivotal for the prevention and treatment of thromboembolic disorders and ˜0.7% of the Western population receives oral anticoagulant treatment (8). Coumarins and heparin are the most well known clinically used anticoagulants. Coumarins inhibit the activity of all vitamin K dependent proteins including procoagulants (thrombin, FXa, FIXa and FVIIa) and anticoagulants (activated protein C (APC) and protein S), whereas heparin mediates its anticoagulant activity by enhancing the inhibition of thrombin and FXa by antithrombin III (9, 10). The non-specific mode of action of these anticoagulants account for their therapeutic limitations in maintaining a balance between thrombosis and hemostasis (11). Hence, there is a need for the development of new anticoagulants, which target specific coagulation enzymes or a particular step in the clotting process (12, 13). Because of its relatively low concentrations in blood (10 nM) and its pivotal role in the initiation of coagulation cascade (14), FVII/FVIIa may be an attractive drug target for the development of novel and specific anticoagulant agents.

Proteins or toxins from snake venoms have been used in the design and development of a number of therapeutic agents or lead molecules, particularly for cardiovascular diseases (15). For example, a family of inhibitors of angiotensin converting enzyme were developed based on bradykinin potentiating peptides from South American snake venoms (16). Inhibitors of platelet aggregation, such as eptifibatide and tirofiban, were designed based on disintegrins, a large family of platelet aggregation inhibitors found in viperid and crotalid snake venoms (17-22). Ancrod extracted from the venom of Malayan pit viper reduces blood fibrinogen levels and has been successfully tested in a variety of ischaemic conditions including stroke (23).

### SUMMARY

Reported herein is the purification and characterization of a three-finger toxin (hemextin A) that mediates anticoagulant activity from the venom of an elapid snake Hemachatus haemachatus (African Ringhals cobra). The anticoagulant activity of hemextin A is enhanced when hemextin A interacts with a second three-finger toxin (hemextin B) to form a complex (hemextin AB complex).

The inventors have shown the formation of a complex between the two proteins may be important for the anticoagulant activity. This is the first tetrameric complex consisting of three-finger toxins. The inventors have shown that hemextin A and its synergistic complex prolongs clotting by inhibiting extrinsic tenase activity using “dissection approach” and by studying their effect on the reconstituted extrinsic tenase complex.

Further, the inventors have confirmed the specificity of hemextin AB complex and hemextin A inhibition by studying their effects on 12 serine proteases. Hemextin AB complex is the first reported natural inhibitor of the FVIIa which does not require a scaffold to mediate its inhibitory activity. Molecular interactions of hemextin AB complex with FVIIa/TF-FVIIa provide a new paradigm in the search for anticoagulants inhibiting the initiation of blood coagulation. The molecular interactions in the formation of hemextin AB complex were also elucidated using biophysical techniques. Based on the results of these studies, a model for this unique anticoagulant complex is proposed as described below.

A first aspect of the invention provides a polypeptide that comprises the amino acid sequence as set forth in SEQ ID NO.1 or SEQ ID NO. 3 or a variant, mutant or fragment thereof.

A second aspect of the invention provides a polypeptide that comprises the amino acid sequence as set forth in SEQ ID NO.2, 4 or 5 or a variant, mutant or fragment thereof. A third aspect of the invention provides a nucleic acid molecule which: (i) encodes a polypeptide according to the first or second aspect of the invention; or (ii) hybridizes to a nucleic acid molecule of part (i) or a variant, mutant, fragment or complement thereof.

A fourth aspect of the invention provides a vector containing a nucleic acid molecule of the third aspect of the invention.

A fifth aspect of the invention provides a host cell transformed with a vector of the fourth aspect of the invention.

A sixth aspect of the invention provides a method of producing a polypeptide according to the first or second aspect of the invention, the method comprising culturing a host cell according to the fifth aspect of the invention under conditions suitable for the expression of the polypeptide of the first or second aspect of the invention.

A seventh aspect of the invention provides a method of producing a polypeptide according to the first or second aspect of the invention, the method comprising the chemical synthesis of the polypeptide.

An eighth aspect of the invention provides a method of generating a complex comprising a polypeptide according to the first aspect of the invention and a polypeptide according to the second aspect of the invention, wherein the method comprises contacting a polypeptide according to the first aspect of the invention with a polypeptide according to the second aspect of the invention under conditions suitable to allow formation of the complex.

A ninth aspect of the invention provides a complex comprising:
(i) a polypeptide of the first aspect of the invention; and
(ii) a polypeptide of the second aspect of the invention. A tenth aspect of the invention provides a method of generating an antibody which recognizes a polypeptide of the first or second aspect of the invention or a complex of the ninth aspect of the invention, wherein the method comprises the steps of:
   (i) immunizing an animal with a polypeptide of the first or second aspect of the invention or a complex of the ninth aspect the invention; and
   (ii) obtaining the antibody from said animal.

An eleventh aspect of the invention provides an antibody which recognizes a polypeptide of the first or second aspect of the invention or a complex of the ninth aspect of the invention.

A twelfth aspect of the invention provides a method of producing an antivenom against a polypeptide according to the first aspect of the invention, a polypeptide according to the second aspect of the invention or a complex according to the ninth aspect of the invention, wherein the method comprises immunizing an animal with a polypeptide according to the first or second aspect of the invention or a complex according to the ninth aspect of the invention and harvesting antibodies from the animal for use in the production of an antivenom.

A thirteenth aspect of the invention provides an antivenom effective against a polypeptide according to the first aspect of the invention, a polypeptide according to the second aspect of the invention or a complex according to the ninth aspect of the invention. A fourteenth aspect of the invention provides a method for identifying a modulator of a polypeptide of the first or second aspect of the invention or a modulator of a complex of the ninth aspect of the invention, wherein the method comprises the steps of:
(i) contacting a test compound with said polypeptide of the first or second aspect of the invention or said complex of the ninth aspect of the invention; and
(ii) determining if the test compound binds to said polypeptide or said complex.

A fifteenth aspect of the invention provides a pharmaceutical composition comprising a polypeptide of the first or second aspect of the invention, a nucleic acid molecule of the third aspect of the invention, a vector of the fourth aspect of the invention, a host cell of the fifth aspect of the invention, a complex of the ninth aspect of the invention, an antibody of the eleventh aspect of the invention, an antivenom of the thirteenth aspect of the invention, or a modulator identified by the method of the fourteenth aspect of the invention.

A sixteenth aspect of the invention provides a polypeptide of the first or second aspect of the invention, a nucleic acid molecule of the third aspect of the invention, a vector of the fourth aspect of the invention, a host cell of the fifth aspect of the invention, a complex of the ninth aspect of the invention, an antibody of the eleventh aspect of the invention, an antivenom of the thirteenth aspect of the invention, or a modulator identified by the method of the fourteenth aspect of the invention for use in medicine.

A seventeenth aspect of the invention provides a combined preparation for use in medicine, the combined preparation comprising:
(i) a polypeptide according to the first aspect of the invention or a nucleic acid molecule encoding the same; and
(ii) a polypeptide according to the second aspect of the invention or a nucleic acid molecule encoding the same.

An eighteenth aspect of the invention provides for the use of a polypeptide of the first or second aspect of the invention, a nucleic acid molecule of the third aspect of the invention, a vector of the fourth aspect of the invention, a host cell of the fifth aspect of the invention or a complex of the ninth aspect of the invention in the manufacture of a medicament for use in treating a patient in need of anticoagulant therapy.

A nineteenth aspect of the invention provides for the use of:
(i) a polypeptide according to the first aspect of the invention or a nucleic acid molecule encoding the same; and
(ii) a polypeptide according to the second aspect of the invention or a nucleic acid molecule encoding the same
   in the manufacture of a combined preparation for treating a patient in need of anticoagulant therapy.

A twentieth aspect of the invention provides a method of treating a patient in need of anticoagulant therapy the method comprising administering to the patient a polypeptide of the first or second aspect of the invention, a nucleic acid molecule of the third aspect of the invention, a vector of the fourth aspect of the invention, a host cell of the fifth aspect, a complex of the ninth aspect of the invention or a pharmaceutical composition of the fifteenth aspect of the invention. A twenty-first aspect of the invention provides a method of treating a patient in need of anticoagulant therapy, the method comprising administering to the patient:
(i) a polypeptide according to the first aspect of the invention or a nucleic acid molecule encoding the same; and
(ii) a polypeptide according to the second aspect of the invention or a nucleic acid molecule encoding the same.

A twenty-second aspect of the invention provides a method of treating snake-bite in a patient, the method comprising administering to the patient a polypeptide of the first or second aspect of the invention, a nucleic acid molecule of the third aspect of the invention, a vector of the fourth aspect of the invention, a host cell of the fifth aspect, a complex of the ninth aspect of the invention or a pharmaceutical composition of the fifteenth aspect of the invention.

A twenty-third aspect of the present invention provides use of a polypeptide of the first or second aspect of the invention, a nucleic acid molecule of the third aspect of the invention, a vector of the fourth aspect of the invention, a host cell of the fifth aspect, a complex of the ninth aspect of the invention or a pharmaceutical composition of the fifteenth aspect of the invention in the manufacture of a medicament for treating snake-bite in a patient.

### BRIEF DESCRIPTION OF DRAWINGS

FIG. 1: Anticoagulant activity of the crude venom. Effect of crude venom on (A) recalcification time and (B) prothrombin time. Note the venom exhibits potent anticoagulant activity in both the assays. Each data point represents the average ±SD.

FIG. 2: Purification of hemextins A and B. (A) Size-exclusion chromatography of the crude venom of H. haemachatus venom on Superdex 30 column. Inset, anticoagulant activity of peak 2 and peak 3. (B) Cation exchange chromatography of peak 3 on Uno S6 column. RP-HPLC profiles of fractions containing hemextins A (C) and B (D) on Jupiter C18 semipreparative column. (E) and (F) capillary liquid chromatography profiles of the hemextins A and B, respectively. The homogeneity and mass of hemextins A and B were determined by ESI-MS. Reconstructed mass spectra of the hemextins A (G) and B (H).

FIG. 3: N-terminal sequences of hemextins A and B. First 37 N-terminal residues of hemextins A and B were determined by Edman degradation. Conserved cysteine residues in the three-finger toxin family are shaded in black. Further sequencing of the proteins resulted in the sequences as set forth in FIG. 13.

FIG. 4: Effects of hemextins A and B on prothrombin time. (A) Effect of hemextins A and B on prothrombin time. Note the anticoagulant potency of hemextin A increases in the presence of hemextin B. Each data point represents the average ±SD. (B) Formation of complex between hemextins A and B is illustrated by their effect on prothrombin time. Each data point represents the average ±SD.

FIG. 5: Gel filtration studies on the formation of hemextin AB complex. Note the elution time of the hemextin AB complex is reduced to ˜40 min over that of the individual hemextins, ˜70 min.

FIG. 6: Localization of the step of activity. (A) Schematic representation showing the selective activation of the extrinsic coagulation pathway by the prothrombin, Stypven and the thrombin time clotting assays. Effect of hemextin A (B), hemextin B (C) and hemextin AB complex (D) on the prothrombin time (Δ); Stypven time () and thrombin time (▪) clotting assays (see text for details). Each data point represents the average ±SD.

FIG. 7: Inhibition of TF-FVIIa activity. (A) The inhibitory potency of hemextin A (), hemextin B (▴) and hemextin AB complex (▪) for the inhibition of FVIIa-TF. (B) Complex formation between hemextins A and B is illustrated by their effect on TF-FVIIa enzymatic activity.

FIG. 8: Effect of phospholipids on the inhibitory activity of hemextins A and B and hemextin AB complex. The inhibitory potency of hemextin A (), hemextin B (▴) and hemextin AB complex (▪) for the inhibition of (A) FVIIa and (B) FVIIa-sTF amidolytic activity. Note the absence of phospholipids do not affect the inhibitory potency of the protein(s) and the reconstituted complex.

FIG. 9: Serine protease specificity. Effect of hemextin A, hemextin B and the hemextin AB complex on the amidolytic activity of (A) FIXa, (B) FXa, (C) FXIa, (D) FXIIa, (E) plasma kallikrein, (F) thrombin, (G) trypsin, (H) chymotrypsin, (I) urokinase, (J) plasmin, (K) APC and (L) tPA. Benzamidine (▪) was used as a positive control in all the experiments except in case of plasmin and chymotrypsin where aprotinin was used. The inhibitory potency of the proteins and the reconstituted complex was measured with respect to the blank (□), an assay mixture containing assay buffer in place of the proteins. Note both hemextin A and hemextin AB complex, but not hemextin B, inhibit the amidolytic activity of plasma kallikrein.

FIG. 10: Inhibition of plasma kallikrein amidolytic activity. The inhibitory potency of hemextin A (≡), hemextin B (▴) and hemextin AB complex (▪) for the inhibition of plasma kallikrein amidolytic activity. Note that the IC₅₀ for the inhibition is ˜5 μM.

FIG. 11: Nature of inhibition. (A) Double reciprocal (Lineweaver-Burk) plots for the kinetic activity of FVIIa-sTF in the presence of 50 nM (□) (2Kᵢ), 25 nM (◯) (Kᵢ), 12.5 nM (▪) (½ Kᵢ) of reconstituted hemextin AB complex. () represents the kinetic activity FVIIa-sTF in the absence of hemextin AB complex. Note that the Vₘₐₓ decreases with increase in the inhibitor concentration where as the Kₘ remains unchanged (see Table 2 for details), a classical phenomenon observed in non-competitive inhibitors. (B) Corresponding secondary plot depicting the Kᵢ for the inhibition. The arrow in the figure depicts the Kᵢ having a value of 25 nM.

FIG. 12: ITC studies on the formation of complex between hemextin AB complex and FVIIa. (A) Raw data in microcalories/s versus time showing heat release upon injections of 0.2 mM of reconstituted hemextin AB complex into a 1.4 mL cell containing 10 μM of FVIIa; (B) Integration of the raw data yields the heat/mol versus molar ratio. The best values of the fitting parameters are 4.11×10⁵ M⁻¹ for K, 7.931 kcal.M⁻¹ for ΔH, and 1.25 cal.M⁻¹ for ΔS.

FIG. 13: Sequence information for Hemextin B and A and sequence comparisons of Hemextin B and A.

FIG. 14: Conformational changes associated with the formation of hemextin complex. CD spectra of (A) hemextin A and (B) hemextin B at various protein concentrations are shown. The conformational changes due to the aggregation at higher concentrations are marked with arrows. (C) Conformational changes in hemextin A with increasing concentrations of hemextin B. (D) CD change in hemextin A at 217 nm with increasing concentrations of hemextin B. Note that no significant changes in CD spectra were observed with further addition of hemextin A after the ratio of hemextin A to hemextin B reached 1:1 (C and D).

FIG. 15. Measurement of molecular diameter during Hemextin AB complex formation using GEMMA. The molecular diameters of the individual hemextins and the hemextin AB complex are calculated based on their electrophoretic mobility. Note the formation of hemextin AB complex leads to an increase in the molecular diameter. Addition of equimolar toxin C does not show any significant increase in the molecular diameters of hemextin A and hemextin B validating the obtained data.

FIG. 16. Measurement of hydrodynamic diameter using DLS. (A) CONTIN analysis hemextin A, hemextin B and hemextin AB complex in 50 mM Tris-HCl buffer. Effect of various concentrations of NaCl (B) and glycerol (C) on hemextin AB complex. The calculated hydrodynamic diameters for each molecular species are shown.

FIG. 17. Interaction studies between hemextin A and B using ITC. (A) Raw ITC data showing heat release upon injections of 1 M hemextin B into a 1.4-ml cell containing 0.1 mM of hemextin A. (B) Integration of the raw ITC data yields the heat/mol versus molar ratio. The best values of the fitting parameters are 1.04 for N, 2.23×10⁶ M⁻¹ for Kₐ and −11.68 kcal.M⁻¹ for ΔH.

FIG. 18. Thermodynamics of hemextin A-hemextin B interaction. (A) Effect of temperature on the energetics of hemextin A-hemextin B interaction: () enthalpy change (ΔH), (▪) change in entropy term (TΔS) and (▴) free energy change (ΔG). (B) Enthalpy-entropy compensation in various protein-protein interactions described in the literature (O) (Data were taken from Ye and Wu (68), McNemar et al. (69) and references cited in the review by Stites (70)) and hemextin A-hemextin B () interactions are shown. Inset shows the enthalpy-entropy compensation in hemextin A-hemextin B interaction.

FIG. 19. Hemextin AB complex formation under different buffer conditions. (A) Effect of buffer ionization on the enthalpy for hemextin AB complex formation. All experiments were performed at pH 7.4. Ionization enthalpy changes used for buffers were 0.71 kcal/mol for phosphate, 5.27 kcal/mol for MOPS, and 11.3 kcal/mol for Tris (Ref). (B) Dependence of Kₐ on the ionic strength of the buffer. The binding affinity decreases with the increase in buffer ionic strength. (C) Dependence of Kₐ on the glycerol concentration. The binding affinity decreases with the increase in glycerol concentration indicating the importance of hydrophobic interactions.

FIG. 20. SEC studies of Hemextin AB complex in different buffer conditions. (A) Elution profiles of hemextin AB complex in Tris-HCl buffer. (B) Tris-HCl buffer of varying ionic strength (by using different concentrations of NaCl). (C) Tris-HCl buffer containing different concentrations of glycerol. The tetrameric complex dissociates into dimer and monomer (peaks denoted by 4, 2 and 1, respectively) with the increase in salt or glycerol. * (D) Calibration for the column using the following proteins as molecular weight markers—(A) ovomucoid (28 kD), (B) ribonuclease (15.6 KD), (C) cytochrome C (12 KD), (D) apoprotinin (7 KD) and (E) pelovaterin (4 KD). The molecular weights of the tetramer, dimer and monomers were calculated from the calibration curve.

FIG. 21. Effect of buffer conditions on anticoagulant activity. Effect of (A) buffer ionic strength on anticoagulant activity and (B) glycerol on anticoagulant activity. The anticoagulant activity of hemextin AB complex decreases with the increase in the buffer ionic strength and also with increase in glycerol concentrations. The arrows indicate the concentrations of (A) salt and (B) glycerol where the anticoagulant complex exists mostly as a mixture of dimer and monomers.

FIG. 22. One-dimensional ¹H NMR studies. Spectrum of (A) hemextin A and (B) hemextin B under different buffer conditions. In the presence of NaCl, the β-sheet structure of hemextin A is completely disrupted.

FIG. 23. A proposed model of hemextin AB complex. (A) Schematic diagram depicting the formation of hemextin AB complex. Hemextins A and B, two structurally similar three-finger toxins, form a compact and rigid tetrameric complex with 1:1 stoichiometry. (B) Schematic diagram showing the effect of salt and glycerol on conformations of hemextins A and B. Hemextin A undergoes a conformational change in the presence of salt. (C) Dissociation of the tetrameric hemextin AB complex in the presence of salt and glycerol. The dissociation probably occurs in two different planes. Thus the hemextin AB dimer in high salt is different from the dimer formed in the presence of glycerol. Two putative anticoagulant sites are shown with dotted semicircles (See text for details).

### DETAILED DESCRIPTION OF THE INVENTION

A first aspect of the invention provides a polypeptide that comprises the amino acid sequence as set forth in SEQ ID NO.1 or SEQ ID NO. 3 or a variant, mutant or fragment thereof.

In one embodiment, the polypeptide consists of the amino acid sequence as set forth in SEQ ID NO.1. In another embodiment, the polypeptide consists of the amino acid sequence as set forth in SEQ ID NO.3.

As discussed below, also comprised herein are functional equivalents of the polypeptides of the first aspect of the invention.

As described herein, Hemextin A exhibits anticoagulant activity on its own. Accordingly, the polypeptide of the first aspect of the invention may exhibit anticoagulant activity.

The polypeptide may in one embodiment be obtained from the venom of H. haemachatus (African Ringhals cobra).

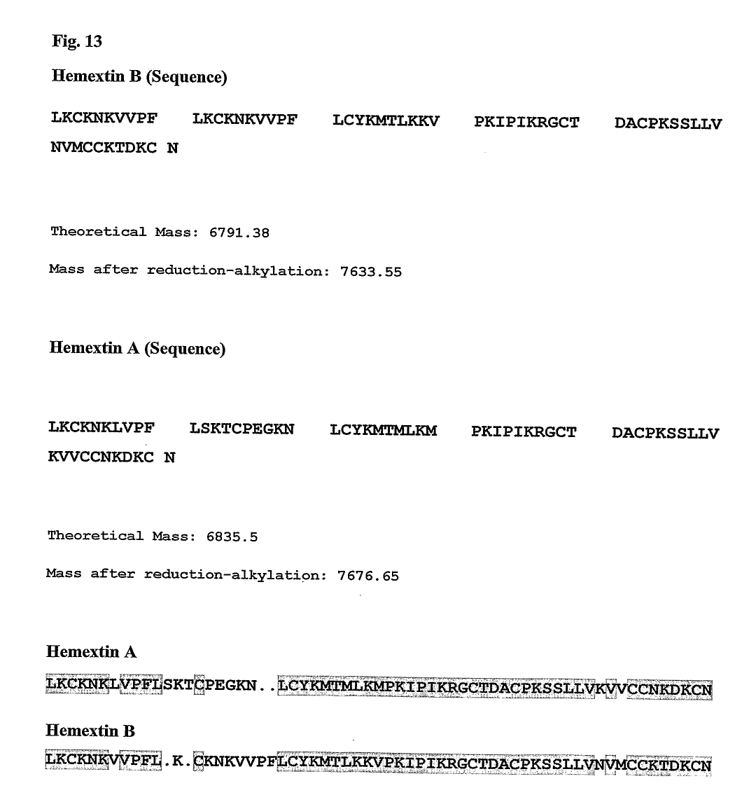

SEQ ID NO.1 is the Hemextin A sequence set forth in FIG. 13, viz:

LKCKNKLVPFLSKTCPEGKNLCYKMTMLKMPKIPIKRGCTDACPKSSLLV
KVVCCNKDKCN

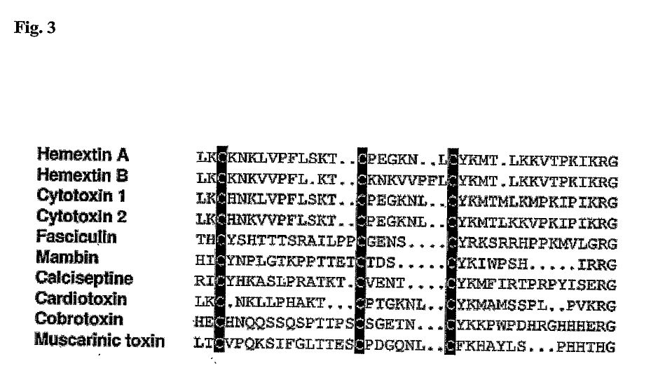

SEQ ID NO.3 is the sequence set forth in the first line of FIG. 3, viz:

LKCKNKLVPFLSKT..CPEGKN..LCYKMT.LKKVTPKIKRG

SEQ ID NO. 3 represents preliminary sequencing results for the N-terminal portion of Hemextin A. Further sequencing of Hemextin A yielded the sequence in SEQ ID NO.1.
(i) A second aspect of the invention provides a polypeptide that comprises the amino acid sequence as set forth in SEQ ID NO.2, 4 or 5 or a variant, mutant or fragment thereof.

In one embodiment, the polypeptide consists of the amino acid sequence as set forth in SEQ ID NO:2. In another embodiment, the polypeptide consists of the amino acid sequence as set forth in SEQ ID NO.4. In yet another embodiment, the polypeptide consists of the amino acid sequence as set forth in SEQ ID NO.5.

As discussed below, also comprised herein are functional equivalents of the polypeptides of the second aspect of the invention. SEQ ID NO.2 is the Hemextin B sequence set forth in FIG. 13, viz:

LKCKNKVVPFLKCKNKVVPFLCYKMTLKKVPKIPIKRGCTDACPKSSLLV
NVMCCKTDKCN

SEQ ID NO.4 is the sequence set forth in the second line of FIG. 3, viz:

LKCKNKVVPFL.KT..CKNKVVPFLCYKMT.LKKVTPKIKRG

SEQ ID NO. 4 represents preliminary sequencing results for an N-terminal portion of Hemextin B.

SEQ ID NO.5 is the Hemextin B sequence set forth in FIG. 13 albeit without the last four amino acids, viz:

LKCKNKVVPFLKCKNKVVPFLCYKMTLKKVPKIPIKRGCTDACPKSSLLV
NVMCCKT

It is believed that there may be variations in the C-terminal portion (in particular the last four amino acids) of SEQ ID NO. 2. Accordingly in one embodiment, there is provided a polypeptide according to the second aspect of the invention which may differ from the sequence set forth in SEQ ID NO.2 at the C-terminal. More specifically, there is provided a polypeptide in which at least one of (e.g. 1, 2, 3 or 4 of) the last four amino acids of SEQ ID NO.2 (e.g. one or more of the first, second, third and/or fourth amino acids at the C-terminal (i.e. DKCN)) differs from that set forth in SEQ ID NO.2.

Hence, in one embodiment of the second aspect of the invention there is provided a polypeptide which comprises SEQ ID NO.5. Since SEQ ID NO.5 is believed to be an incomplete sequence of Hemextin B, then in one embodiment there is provided a polypeptide which comprises SEQ ID NO:5 and one or more additional amino acids (e.g. 1, 2, 3, 4, 5, 6 etc.) at the C-terminal end of the amino acid sequence of SEQ ID NO.5.

The polypeptide of the second aspect of the invention may in one embodiment be obtained from the venom of H. haemachatus (African Ringhals cobra).

The polypeptide according to the second aspect of the invention can form a complex with a polypeptide according to the first aspect of the invention such that there is a synergistic effect on the anticoagulant activity of the polypeptide according to the first aspect of the invention.

The polypeptides of the first and second aspects of the present invention are not necessarily physically derived from the snake venom but may be generated in any manner, including for example, by recombinant technology and by chemical synthesis such as by solid-phase peptide synthesis. In an alternative embodiment, there is provided a protein according to the first or second aspect of the invention which is purified from the snake venom of H. haemachatus. Methods for purifying polypeptides are well known in the art and may be used to purify a polypeptide of the first or second aspects of the invention. Purification of the polypeptides may also be achieved as described in the Examples section herein. Thus, in one embodiment the polypeptides of the first and second aspect are obtained or are obtainable by the method described in the Examples section herein.

The polypeptides of the first and second aspects of the present invention may be in their naturally occurring form, albeit isolated from their native environment, or may be modified, provided that they retain the functional characteristic of exhibiting anticoagulant activity either alone (e.g. in the case of polypeptides of the first aspect of the invention) or when in the form of the complex of the invention. For example, the polypeptides may be modified chemically to introduce one or more chemical modifications to the amino acid structure.

With regard to determination or verification of protein and nucleic acid sequences, persons skilled in the art will appreciate that where a partial amino acid sequence of a polypeptide is known, oligonucleotide probes can be designed to probe a genomic or cDNA library of H. haemachatus and to thereby determine or verify the polypeptide or gene sequences of interest. Since the genetic code is redundant, multiple nucleotide sequences can encode the same peptide sequence. To be sure that the actual nucleotide sequence is present in a probe oligonucleotide, the oligonucleotide is synthesized incorporating, where needed, multiple nucleotides.

Methods for designing, creating and using degenerate probes are well known in the art. See for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Methods of using degenerate probes for elucidating gene sequences and the encoded polypeptides are also well known in the art and can be readily accomplished by the skilled person.

The polypeptides of the first and second aspects of the invention may form a complex with each other such that there is a synergistic effect on the anticoagulant activity of the polypeptide according to the first aspect of the invention. Accordingly, in an embodiment of the first and second aspects of the invention there is provided a complex comprising a polypeptide of the first aspect of the invention and a polypeptide of the second aspect of the invention. As discussed below, the complex is believed to be a tetramer. In one embodiment, the complex may be a heterodimer. Such complexes may be used in the various aspects of the invention, e.g. in the treatment of patients in need of anti-coagulant therapy.

The polypeptide according to the first aspect of the invention may have a molecular weight which is determined as being about 6835.00±50, 20, 10, 15, 10, 5, 2, 1 or 0.52 daltons.

The polypeptide according to the first aspect of the invention may have a molecular weight which is determined as being about 6835.50±50, 20, 10, 15, 10, 5, 2, 1 or 0.52 daltons.

The polypeptide according to the second aspect of the invention may have a molecular weight which is determined as being about 6791.38±50, 20, 10, 15, 10, 5, 2, 1 or 0.32 daltons.

The polypeptide according to the second aspect of the invention may alternatively have a molecular weight which is determined as being about 6792.56±50, 20, 10, 15, 10, 5, 2, 1 or 0.32 daltons.

The method used in the Examples section may, for example, be used to determine molecular weight. Other methods known in the art may alternatively be used.

The terms “polypeptide” and “protein” are used interchangeably and refer to any polymer of amino acids (dipeptide or greater) linked through peptide bonds or modified peptide bonds, whether produced naturally or synthetically. Polypeptides of less than about 10-20 amino acid residues are commonly referred to as “peptides.”

The polypeptides of the invention may also comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

The term “comprising” and grammatical variants thereof as used herein means “including”. Thus, for example, a composition “comprising” X may consist exclusively of X or may include one or more additional components. Similarly, a polypeptide molecule comprising a given sequence may consist exclusively of the given sequence or may include one or more additional components. For instance, the polypeptides of the invention may comprise one or more additional amino acids at their N or C termini.

The polypeptides of the first and second aspects of the invention include variants of the recited sequences. Such variant sequences may include, for example, allelic variants or variant sequences identified as a result of further sequencing studies on Hemextin A or Hemextin B. Also included are functional equivalents, active fragments and fusion proteins. For the avoidance of doubt, the first and second aspects of the invention include: functional equivalents of the variants and active fragments of the variants. Also included are fusion proteins comprising the variants, functional equivalents and active fragments. Similarly, the invention extends to variants and active fragments of the functional equivalents.

The polypeptide of the first aspect of the invention and the polypeptide of the second aspect of the invention may be provided in the form of a complex with a polypeptide of the second aspect of the invention or a polypeptide of the first aspect of the invention respectively. The polypeptide of the first aspect of the invention may be hemextin A, a variant, mutant, functional equivalent or active fragment of hemextin A or a fusion protein comprising hemextin A. The polypeptide of the second aspect of the invention may be hemextin B, a variant, mutant, functional equivalent or active fragment of hemextin B or a fusion protein comprising hemextin B. Hence, various combinations of hemextin A and hemextin B, variants, mutants, functional equivalents, active fragments, and fusion proteins of hemextin A and hemextin B are envisaged providing that the resulting complex possesses anticoagulant activity.

In one embodiment, a polypeptide or polypeptide complex is deemed to exhibit anticoagulant activity if it increases the prothrombin time or if it inhibits the activity of the extrinsic tenase activity.

To determine if a polypeptide or polypeptide complex exhibits anticoagulant activity the prothrombin test may be employed as described below in the Examples section. Briefly, prothrombin times may be measured according to the method of Quick (see Quick A J. (1935) J. Biol. Chem. 109, 73-74). 100 μl of 50 mM of Tris-HCl buffer (pH 7.4), 100 μl of plasma and 50 μL of the protein under investigation are to be incubated for 2 min at 37° C. Clotting is initiated by the addition of 150 μL of thromboplastin with calcium reagent. If the polypeptide exhibits anticoagulant activity, the prothrombin time will increase.

Alternatively or additionally, the effect of the polypeptide or polypeptide complex on extrinsic tenase activity can be assessed as described below in the Examples section. As discussed herein hemextin A and its complex with hemextin B is believed to inhibit the activation of FX by the TF-FVIIa complex (the extrinsic tenase complex). Thus, a polypeptide according to the first aspect of the invention and a complex formed from a polypeptide according to the first and second aspects of the invention suitably inhibit the ability of the TF-FVIIa complex to catalyse the activation of FX to FXa. Details of how this may be determined are set forth in the Examples section below where it is described how the inhibitory effect of individual proteins and the complex on extrinsic tenase activity can be determined by measuring the effect of the protein or complex on FXa formation.

In one embodiment a variant, mutant, functional equivalent or active fragment of hemextin A is capable of at least about 20%, 30%, 40%, 50%, 55%, 60%, 65%, 70%, 75%, 80%, 85%, 90% or 95% inhibition of extrinsic tenase activity when in the form of a complex with hemextin B or with a variant, mutant, functional equivalent or active fragment of hemextin B.

In one embodiment a variant, mutant, functional equivalent or active fragment of hemextin B is capable of at least about 20%, 30%, 40%, 50%, 55%, 60%, 65%, 70%, 75%, 80%, 85%, 90% or 95% inhibition of extrinsic tenase activity when in the form of a complex with hemextin A or with a variant, mutant, functional equivalent or active fragment of hemextin A.

To determine whether a putative functional equivalent, active fragment or fusion protein of hemextin A is capable of forming a synergistic complex with a polypeptide according to the second aspect of the invention or to determine the extent of anticoagulant activity, hemextin B is optionally used.

Likewise, to determine whether a putative variant, mutant, functional equivalent or active fragment of hemextin B is capable of forming a synergistic complex with a polypeptide according to the first aspect of the invention or to determine the extent of anticoagulant activity, hemextin A is optionally used.

Variants include, for example, allelic variants within the species from which the polypeptides are derived. Additionally, it is possible that the last four amino acids of SEQ ID NO. 2 may be subject to variation. Accordingly, the identification of sequences which are variant sequences of SEQ ID NO. 1, 2, 3, 4 or 5 and which may be identified as a result of further sequencing studies on Hemextin A or B are also included within the scope of the first and second aspects of the invention.

The variants of the invention may include polypeptides in which one or more of the amino acid residues are substituted with one or more conserved or non-conserved amino acid residues (preferably a conserved amino acid residue). Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

Particularly preferred are variants in which several, for example, between 5 and 10, 1 and 5, 1 and 3, 1 and 2 or just 1 amino acids are substituted, deleted or added in any combination. Especially preferred are silent substitutions, additions and deletions, which do not alter the properties and activities of the polypeptide. Also especially preferred in this regard are conservative substitutions. Variant or “mutant” polypeptides also include polypeptides in which one or more of the amino acid residues include a substituent group. Variants are also contemplated where it is desirable to modify an amino acid sequence such as to modify the properties of the polypeptide, for instance its biological activity.

Further embodiments of the first and second aspects of the invention provide functional equivalents of the polypeptides of the invention that contain single or multiple amino-acid substitution(s), addition(s), insertion(s) and/or deletion(s) and/or substitutions of chemically-modified amino acids, wherein “functional equivalent” denotes a polypeptide that: (i) possesses the functional characteristic of exhibiting anticoagulant activity either alone or when in the form of the complex; or (ii) which has an antigenic determinant in common with the polypeptide.

A functionally-equivalent polypeptide according to this aspect of the invention may be a polypeptide that has at least 60% sequence identity to a polypeptide of the invention. In one embodiment, there is provided a functionally-equivalent polypeptide that has at least 60% sequence identity with hemextin A, hemextin B or an allelic variant thereof.

Methods of measuring protein sequence identity are well known in the art and it will be understood by those of skill in the art that in the present context, sequence identity is calculated on the basis of amino acid identity (sometimes referred to as “hard homology”). For example the UWGCG Package provides the BESTFIT program which can be used to calculate sequence identity (for example used on its default settings) (Devereux et al (1984) Nucleic Acids Research 12, p 387-395). The PILEUP and BLAST algorithms can be used to calculate sequence identity or line up sequences (typically on their default settings), for example as described in Altschul S. F. (1993) J Mol Evol 36:290-300; Altschul, S, F et al (1990) J Mol Biol 215:403 Software for performing BLAST analyses is publicly available through the National Center for Biotechnology Information on the world wide web through the internet at, for example, “www.ncbi.nlm.nih.gov/”. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. The BLAST algorithm performs a statistical analysis of the similarity between two sequences; see e.g., Karlin and Altschul (1993) Proc. Nad. Acad. Sci. USA 90: 5873 One measure of similarity provided by the BLAST algorithm is the smallest sum probability (P(N)), which provides an indication of the probability by which a match between two nucleotide or amino acid sequences substituted for each other.

Typically, greater than 60% sequence identity between two polypeptides is considered to be an indication of functional equivalence, provided that either the functional characteristic of the polypeptide in exhibiting anticoagulant activity either alone or when in the form of the complex of the polypeptide is present or the polypeptide possesses an antigenic determinant in common with the polypeptide. In one embodiment, a functionally equivalent polypeptide according to this aspect of the invention exhibits a degree of sequence identity with the polypeptide, or with a fragment thereof, of greater than 60%. The polypeptides may have a degree of sequence identity of greater than 70%, 80%, 90%, 95%, 97%, 98% or 99%, respectively.

Functionally-equivalent polypeptides according to the invention are therefore intended to include mutants (such as mutants containing amino acid substitutions, insertions or deletions). Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the polypeptide. Also especially preferred in this regard are conservative substitutions. “Mutant” polypeptides also include polypeptides in which one or more of the amino acid residues include a substituent group.

Functional equivalents with improved function may also be designed through the systematic or directed mutation of specific residues in the polypeptide sequence.

Also included within the scope of the first and second aspects of the invention are active fragments wherein “active fragment” denotes a truncated polypeptide that: (i) possesses the functional characteristic of exhibiting anticoagulant activity either alone or when in the form of the complex or (ii) which has an antigenic determinant in common with the polypeptide.

Active fragments of the invention comprise at least n consecutive amino acids from a polypeptide of the invention. Suitably, the active fragment comprises at least n consecutive amino acids from SEQ ID NO. 1, SEQ ID NO.2, SEQ ID NO. 3, SEQ ID NO. 4 or SEQ ID NO. 5 or a variant, mutant or functional equivalent of any one of these sequences etc. n typically is 7 or more (for example, 8, 10, 12, 14, 16, 18, 20, 25, 35, 40, 45, 50, 55 or 60 or more).

The polypeptides of the invention (e.g. the variants, mutants, functional equivalents or fragments of the polypeptides of the invention) may be “free-standing”, i.e. not part of or fused to other amino acids or polypeptides, or they may be comprised within a larger polypeptide of which they form a part or region. When comprised within a larger polypeptide, the polypeptide of the invention in one embodiment forms a single continuous region. Additionally, several polypeptides may be comprised within a single larger polypeptide.

In one embodiment of the first and second aspects of the invention, there is provided a functional equivalent or an active fragment which has an antigenic determinant in common with a polypeptide of the invention. In one embodiment, the antigenic determinant is shared with hemextin A, hemextin B or an allelic variant thereof. In one embodiment, the antigenic determinant is shared with SEQ ID NO. 1, SEQ ID NO.2, SEQ ID NO.3, SEQ ID NO.4 or SEQ ID NO:5.

“Antigenic determinant” refers to a fragment of a molecule (i.e., an epitope) that makes contact with a particular antibody. “Antigenic determinants” or epitopes usually consist of chemically active surface groupings of molecules such as amino acids or sugar side chains and have specific three dimensional structural characteristics as well as specific charge characteristics.

It is known in the art that relatively short synthetic peptides that can mimic antigenic determinants of a protein can be used to stimulate the production of antibodies against the protein (see, for example, Sutcliffe et al., Science 219:660 (1983)). Antigenic epitope-bearing peptides and polypeptides can contain, for example, at least four to ten amino acids, at least ten to 15 amino acids, or about 15 to about 25 amino acids. Such epitope-bearing peptides and polypeptides can be produced by fragmenting the protein, or by chemical peptide synthesis, as described herein.

Moreover, antigenic determinants can be selected by phage display of random peptide libraries (see, for example, Lane and Stephen, Curr. Opin. Immunol. 5:268 (1993), and Cortese et al., Curr. Gpin. Biotechnol. 7.616 (1996)). Standard methods for identifying antigenic determinants and producing antibodies from small peptides that comprise an antigenic determinant are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies Production, Engineering, and Clinical Application, Ritter and Ladyman (eds.), pages 6084 (Cambridge University Press 1995), and Coligan et al. (eds.), Current Protocols in Immunology, pages 91-95 and pages 91-911 (John Wiley & Sons 1997).

Such polypeptides possessing an antigenic determinant can be used to generate ligands, such as polyclonal or monoclonal antibodies, that are immunospecific for the polypeptides of the invention. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

In one embodiment of the first and second aspect of the invention, there is provided a fusion protein comprising a polypeptide of the invention fused to a peptide or other polypeptide, such as a label, which may be, for instance, bioactive, radioactive, enzymatic or fluorescent, or an antibody.

For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Fusion proteins may also be useful to screen peptide libraries for inhibitors of the activity of the polypeptides of the invention. It may be useful to express a fusion protein that can be recognised by a commercially-available antibody. A fusion protein may also be engineered to contain a cleavage site located between the sequence of the polypeptide of the invention and the sequence of a heterologous polypeptide so that the polypeptide may be cleaved and purified away from the heterologous polypeptide. By a “heterologous polypeptide”, we include a polypeptide which, in nature, is not found in association with a polypeptide of the invention.

In a preferred embodiment of the first and second aspect of the invention there is provided a polypeptide which comprises the amino acid sequence as set forth in SEQ ID NO. 1, 2, 3, 4 or 5 (and preferably as set forth in SEQ ID NO. 1, 2 or 5) or a variant, mutant, functional equivalent or active fragment thereof. In one embodiment the polypeptide consists of the amino acid sequence as set forth in SEQ ID NO. 1, 2, 3, 4, 5 or a variant, mutant, functional equivalent or active fragment thereof. It will be appreciated that the polypeptides of the invention (e.g. SEQ ID NOs.1, 2, 3, 4 or 5) may, for example, find utility in raising antibodies against hemextin A and hemextin B.

A third aspect of the invention provides a nucleic acid molecule which: (i) encodes a polypeptide according to the first or second aspect of the invention; or (ii) hybridizes to a nucleic acid molecule of part (i) or a variant, mutant, fragment or complement thereof.

The oligonucleotide may be a primer or a probe. The oligonucleotide may comprise a region of nucleotide sequence that hybridizes under stringent conditions to at least 10, 12, 15, 17, 20, 25, 30, 35 or 40 consecutive nucleotides of a nucleic acid molecule according to (i) In one embodiment of the third aspect of the invention the nucleic acid molecule is a probe or a primer comprising an oligonucleotide, which oligonucleotide comprises a region of nucleotide sequence that hybridizes under stringent conditions to at least 10, 12, 15, 17, 20, 25, 30, 35 or 40 consecutive nucleotides of a nucleic acid molecule (and preferably a naturally occurring nucleic acid molecule) encoding SEQ ID NO. 1, 2, 3, 4 or 5 (or a variant, mutant or functional equivalent or active fragment thereof etc.). In one embodiment, the nucleic acid molecule is a probe or a primer comprising an oligonucleotide, which oligonucleotide comprises a region of nucleotide sequence which is complementary to at least 10, 12, 15, 17, 20, 25, 30, 35 or 40 consecutive nucleotides of a nucleic acid molecule (and preferably a naturally occurring nucleic acid molecule) encoding a polypeptide of the first aspect of the invention, for example, a polypeptide as set forth in SEQ ID NO. 1, 2, 3, 4 or 5 (or a variant, mutant, functional equivalent or active fragment thereof etc.).

The stringent conditions may be low stringency, medium stringency, medium/high stringency, high stringency or very high stringency.

It will be appreciated by those skilled in the art that as a result of the degeneracy of the genetic code, a multitude of nucleic acid molecules encoding the polypeptides of the first and second aspects of the invention, some bearing minimal sequence identity to the polynucleotide sequences of any known and naturally occurring gene, may be produced. Thus, the invention contemplates each and every possible variation of polynucleotide sequence that could be made by selecting combinations based on possible codon choices.

Moreover, those skilled in the art will appreciate that codons may be selected to increase the rate at which expression of the peptide or polypeptide occurs in a particular prokaryotic or eukaryotic host in accordance with the frequency with which particular codons are utilized by the host.

Nucleic acids of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

The term “nucleic acid molecule” also includes analogues of DNA and RNA, such as those containing modified backbones, for example, peptide nucleic acids.

Suitable experimental conditions for determining whether a given nucleic acid molecule hybridises to a specified nucleic acid may involve presoaking of a filter containing a relevant sample of the nucleic acid to be examined in 5×SSC for 10 min, and prehybridisation of the filter in a solution of 5×SSC, 5×Denhardt's solution, 0.5% SDS and 100 μg/mL of denatured sonicated salmon sperm DNA, followed by hybridisation in the same solution containing a concentration of 10 ng/mL of a 32P-dCTP-labeled probe for 12 hours at approximately 45° C., in accordance with the hybridisation methods as described in Sambrook et al. (1989; Molecular Cloning, A Laboratory Manual, 2nd edition, Cold Spring Harbour, New York).

The filter is then washed twice for 30 minutes in 2×SSC, 0.5% SDS at least 55° C. (low stringency), at least 60° C. (medium stringency), at least 65° C. (medium/high stringency), at least 70° C. (high stringency), or at least 75° C. (very high stringency). Hybridisation may be detected by exposure of the filter to an X-ray film.

Further, there are numerous conditions and factors, well known to those skilled in the art, which may be employed to alter the stringency of hybridisation. For instance, the length and nature (DNA, RNA, base composition) of the nucleic acid to be hybridised to a specified nucleic acid; concentration of salts and other components, such as the presence or absence of formamide, dextran sulfate, polyethylene glycol etc; and altering the temperature of the hybridisation and/or washing steps.

Further, it is also possible to theoretically predict whether or not two given nucleic acid sequences will hybridise under certain specified conditions. Accordingly, as an alternative to the empirical method described above, the determination as to whether a variant nucleic acid sequence will hybridise can be based on a theoretical calculation of the Tm (melting temperature) at which two heterologous nucleic acid sequences with known sequences will hybridise under specified conditions, such as salt concentration and temperature.

In determining the melting temperature for heterologous nucleic acid sequences (Tₘ₍ₕₑₜₑᵣₒ₎) it is necessary first to determine the melting temperature (Tₘ₍ₕₒₘₒ₎) for homologous nucleic acid sequence. The melting temperature (Tₘ₍ₕₒₘₒ₎) between two fully complementary nucleic acid strands (homoduplex formation) may be determined in accordance with the following formula, as outlined in Current Protocols in Molecular Biology, John Wiley and Sons, 1995, as:

Tₘ₍ₕₒₘₒ₎=81.5° C.+16.6(log M)+0.41(%GC)−0.61(% form)−500/L
M=denotes the molarity of monovalent cations,
% GC=% guanine (G) and cytosine (C) of total number of bases in the sequence,
% form=% formamide in the hybridisation buffer, and
L=the length of the nucleic acid sequence.

Tₘ determined by the above formula is the Tₘ of a homoduplex formation (Tₘ₍ₕₒₘₒ₎) between two fully complementary nucleic acid sequences. In order to adapt the Tₘ value to that of two heterologous nucleic acid sequences, it is assumed that a 1% difference in nucleotide sequence between two heterologous sequences equals a 1° C. decrease in Tₘ. Therefore, the Tₘ₍ₕₑₜₑᵣₒ₎ for the heteroduplex formation is obtained through subtracting the sequence identity % difference between the analogous sequence in question and the nucleotide probe described above from the Tₘ₍ₕₒₘₒ₎.

The polypeptides, nucleic acid molecules and antibodies of the present invention are “purified”. The term purified as used herein means altered “by the hand of man” from its natural state; i.e., if it occurs in nature, it has been changed or removed from its natural host or environment. Associated impurities may be reduced or eliminated. In one embodiment, the object species is the predominant species present (i.e., on a molar basis it is more abundant than any other individual species in the composition). In one embodiment, the object species is present in a substantially purified fraction. A substantially purified fraction includes a composition wherein the object species comprises at least about 30 percent (on a molar basis) of all macromolecular species present. Generally, a substantially pure composition will comprise more than about 80 to 90 percent of all macromolecular species present in the composition. In one embodiment, the object species is purified to essential homogeneity (contaminant species cannot be detected in the composition by conventional detection methods) wherein the composition consists essentially of a single macromolecular species.

The nucleic acid molecule may be provided in the form or a “naked” nucleic acid molecule, vector or host cell comprising the same. See the fourth and fifth aspects of the invention in this regard. Where the nucleic acid molecule is for administration to a patient, the general guiding principle is that the nucleic acid molecule upon administration to the patient should be such that the polypeptide may be expressed by the nucleic acid molecule. This will be readily achievable by persons skilled in the art and considerations will include the presence of appropriate regulatory elements such as promoters etc.

A fourth aspect of the invention provides a vector, such as an expression vector, that contains a nucleic acid molecule of the third aspect of the invention. The vectors of the present invention may comprise a transcription promoter, and a transcription terminator, wherein the promoter is operably linked with the nucleic acid molecule, and wherein the nucleic acid molecule is operably linked with the transcription terminator. The vectors may further comprise ribosomal binding sites, translational start and stop sequences, and enhancer or activator sequences. Many prokaryotic and eukaryotic expression vectors are commercially available. Selection of appropriate expression vectors is within the knowledge of those having skill in the art.

In one embodiment, the vector comprises a nucleic acid sequence encoding a polypeptide according to the first aspect of the invention.

In one embodiment, the vector comprises a nucleic acid sequence encoding a polypeptide according to the second aspect of the invention.

In one embodiment, the vector comprises a nucleic acid sequence encoding a polypeptide according to the first aspect of the invention and a nucleic acid sequence encoding a polypeptide according to the second aspect of the invention.

The vectors of the present invention may comprise further genes such as marker genes which allow for the selection of said vector in a suitable host cell and under suitable conditions.

The present invention further includes recombinant host cells comprising these vectors and expression vectors. Hence, a fifth aspect of the invention provides a host cell transformed with a vector of the fourth aspect of the invention. Illustrative host cells include bacterial, yeast, fungal, insect, avian, mammalian, and plant cells.

In one embodiment, there is provided a host cell transformed with a vector according to the fourth aspect of the invention such that a polypeptide of the first aspect of the invention may be expressed by the host cell.

In one embodiment, there is provided a host cell transformed with a vector according to the fourth aspect of the invention such that a polypeptide of the second aspect of the invention may be expressed by the host cell.

In one embodiment, there is provided a host cell transformed with a vector according to the fourth aspect of the invention such that a polypeptide of the first aspect of the invention is expressed by the host cell and a polypeptide of the second aspect of the invention may be expressed by the host cell. The polypeptides of the first and second aspects of the invention may be encoded by different vectors in which case the host cell may be transformed with at least two different vectors according to the fourth aspect of the invention.

A sixth aspect of the invention provides a method of producing a polypeptide according to the first or second aspect of the invention, the method comprising culturing a host cell according to the fifth aspect of the invention under conditions suitable for the expression of the polypeptide of the first or second aspect of the invention.

In one embodiment, the host cell expresses a polypeptide according to the first aspect of the invention.

In another embodiment, the host cell expresses a polypeptide according to the second aspect of the invention.

In yet another embodiment, the host cell expresses a polypeptide according to the first and second aspects of the invention.

A seventh aspect of the invention provides a method of producing a polypeptide according to the first or second aspect of the invention the method comprising the chemical synthesis of the polypeptide. Chemical synthesis may be achieved by, for example, solid-phase peptide synthesis. Such techniques are well known in the art and will be readily able to be carried out by the skilled person.

The methods of the sixth and seventh aspect of the invention may further comprise purifying the polypeptide. Such methods are well known in the art and can be readily performed by the skilled person.

As mentioned above, the polypeptides of the first and second aspects of the invention may be provided in the form of a complex comprising a polypeptide according to the first aspect of the invention and a polypeptide according to the second aspect of the invention. Suitably, the complex is a tetramer.

Accordingly, an eighth aspect of the invention provides a method of generating a complex which comprises a polypeptide according to the first aspect of the invention and a polypeptide according to the second aspect of the invention wherein the method comprises contacting a polypeptide according to the first aspect of the invention with a polypeptide according to the second aspect of the invention under conditions suitable to allow formation of the complex.

Persons skilled in the art will readily be able to determine suitable conditions to allow formation of the complex. Moreover, as indicated in the Examples section below suitable conditions include incubating equimolar concentration of a polypeptide according to the first aspect of the invention and a polypeptide according to the second aspect of the invention at 37° C. for a period of five min in 50 mM Tris-buffer (pH 7.4).

A ninth aspect of the invention provides a complex comprising a polypeptide of the first aspect of the invention and a polypeptide of the second aspect of the invention.

Preferably, the polypeptide of the first aspect and second aspects of the invention are present in a ratio of 1:1.

Preferably, the complex is a tetramer of the two polypeptides.

In one embodiment, the complex is obtained by the method of the eighth aspect of the invention.

As mentioned above, the complex is believed to be in the form of a tetramer.

In one embodiment, the polypeptide according to the first aspect of the invention is hemextin A.

In one embodiment, the polypeptide according to the second aspect of the invention is hemextin B.

Whilst the polypeptides in the complex may be hemextin A and hemextin B, it will be appreciated from the foregoing discussion that one or both of the polypeptides may be a variant, mutant, functional equivalent, active fragment or fusion polypeptide as described above. A tenth aspect of the invention provides a method of generating an antibody which recognizes a polypeptide of the first or second aspect of the invention or a complex of the ninth aspect of the invention, wherein the method comprises the steps of:
(i) immunizing an animal with a polypeptide of the first or second aspect of the invention or a complex of the ninth aspect of the invention; and
(ii) obtaining the antibody from said animal.

An eleventh aspect of the invention provides an antibody which recognizes a polypeptide of the first or second aspect of the invention.

In one embodiment, the antibody binds to hemextin A or B. In one embodiment, the antibody binds to an epitope comprised within the sequence of SEQ ID NO. 1, 2, 3, 4, or 5.

In one embodiment, the antibody of the tenth and eleventh aspects of the invention recognise an antigenic determinant on a polypeptide according to the first or second aspect of the invention which antigenic determinant is exposed when the polypeptide forms a complex with a polypeptide according to the other aspect of the invention. Hence, in one embodiment the antibody recognizes a complex formed by a polypeptide according to the first aspect of the invention and a polypeptide according to the second aspect of the invention. Such antibodies may be raised by using the complex as an immunogen.

The antibodies of the invention may be polyclonal or monoclonal antibody preparations, monospecific antisera, human antibodies, or may be hybrid or chimeric antibodies, such as humanized antibodies, altered antibodies (Fab′)₂ fragments, F(ab) fragments, Fv fragments, single-domain antibodies, dimeric or trimeric antibody fragments or constructs, minibodies, or functional fragments thereof which bind to the antigen in question.

Antibodies may be produced using techniques well known to those of skill in the art and disclosed in, for example, U.S. Pat. Nos. 4,011,308; 4,722,890; 4,016,043; 3,876,504; 3,770,380; and 4,372,745. See also Antibodies—A Laboratory Manual, Harlow and Lane, eds., Cold Spring Harbor Laboratory, N.Y. (1988). For example, polyclonal antibodies are generated by immunizing a suitable animal, such as a mouse, rat, rabbit, sheep, or goat, with an antigen of interest. In order to enhance immunogenicity, the antigen can be linked to a carrier prior to immunization. Such carriers are well known to those of ordinary skill in the art. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies may be prepared using the method of Kohler & Milstein (1975) Nature 256:495-497, or a modification thereof. Typically, a mouse or rat is immunized as described above. Rabbits may also be used. However, rather than bleeding the animal to extract serum, the spleen (and optionally several large lymph nodes) is removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Humanized and chimeric antibodies are also useful in the invention. Hybrid (chimeric) antibody molecules are generally discussed in Winter et al. (1991) Nature 349: 293-299 and U.S. Pat. No. 4,816,567. Humanized antibody molecules are generally discussed in Riechmann et al. (1988) Nature 332:323-327; Verhoeyan et al. (1988) Science 239:1534-1536; and U.K. Patent Publication No. GB 2,276,169, published 21 Sep. 1994).

An antibody is said to “recognize” a molecule if it is capable of specifically reacting with the molecule to thereby bind the molecule to the antibody. The antibodies of the invention may be provided in the form of antibody already bound to a polypeptide of the invention or may be provided in the form of antibody which is not bound to a polypeptide of the invention.

In one embodiment, the antibody or fragment thereof has binding affinity or avidity greater than about 10⁵ M⁻¹, more preferably greater than about 10⁶ M⁻¹, more preferably still greater than about 10⁷ M⁻¹ and most preferably greater than about 10⁸ M⁻¹ or 10⁹ M⁻¹. The binding affinity of an antibody can be readily determined by one of ordinary skill in the art, for example, by Scatchard analysis (Scatchard, Ann. NY Acad. Sci. 51:660 (1949)).

A twelfth aspect of the invention provides a method of producing an antivenom against a polypeptide according to the first aspect of the invention, a polypeptide according to the second aspect of the invention or a complex according to the ninth aspect of the invention, wherein the method comprises immunizing an animal with a polypeptide according to the first or second aspect of the invention or a complex according to the ninth aspect of the invention and harvesting antibodies from the animal for use in the production of an antivenom.

The animal may be immunized with a polypeptide according to the first aspect of the invention or a polypeptide according to the second aspect of the invention or both, either provided separately (as separate or combined preparations) or in the form of a complex of the two polypeptides.

Traditional methods of producing the antivenom is to immunize a mammal such as a horse, goat or sheep against the venom. To reduce their toxicity, the venoms may be modified by treatment with formalin. To prolong their absorption, the modified venoms may be mixed with aluminum hydroxide gel. The antibodies thus produced are then isolated from the animal and used as an antidote in the patient, typically a human patient. More recently, non-mammals have employed using birds such as chickens. In this procedure, young chickens are immunized with small doses of the target-snake venom and as these animals grow older they develop antibodies which act as antidotes against the toxin. As the chickens become hens and start egg production, it has been found that the antivenom proteins are passed on, accumulating in the yolk. The eggs are then harvested for extraction of the proteins used to make the antidote.

The serum of the first animal (e.g. horse or chicken) is then administered to the afflicted animal (the “host”) to supply a source of specific and reactive antibody. The administered antibody functions to some extent as though it were endogenous antibody, binding the venom toxins and reducing their toxicity.

A thirteenth aspect of the invention provides an antivenom effective against a polypeptide according to the first aspect of the invention, a polypeptide according to the second aspect of the invention or a complex according to the ninth aspect of the invention. The antivenom may be produced in accordance with the twelfth aspect of the invention but the method of the fourteenth aspect of the invention may alternatively be used.

A fourteenth aspect of the invention provides a method for identifying a modulator of a compound of a polypeptide of the first or second aspect of the invention or a modulator of a complex of the ninth aspect of the invention.

The terms “modulator” and “modulates” etc. as used herein refer to compounds which are antagonists, agonists or which can function as both antagonists and agonists. For the avoidance of doubt, it will be understood that “modulator” includes compounds that are capable of increasing the anticoagulant activity of a polypeptide or complex of the invention and also includes compounds that are capable of decreasing the anticoagulant activity of a polypeptide or complex of the invention.

The polypeptides of the first and second aspects of the invention can be used to screen libraries of compounds in any of a variety of drug screening techniques. Such compounds may modulate the activity of a polypeptide of the first or second aspect of the invention or a complex of the two polypeptides of the invention.

In one embodiment, the method comprises contacting a test compound with a polypeptide of the first or second aspect of the invention and determining if the test compound binds to the polypeptide of the first or second aspect of the invention. The polypeptide may be provided in the form of a complex comprising the two polypeptides of the invention. The method may further comprise determining if the test compound enhances or decreases the activity of a polypeptide of the first or second aspect of the invention or enhances or decreases the activity of a complex of the two polypeptides of the invention.

By the activity of a polypeptide of the first or second aspect of the invention, we include: (i) the activity of the polypeptide as an anticoagulant when the polypeptide is on its own (e.g. in the case of the polypeptide of the first aspect of the invention); (ii) its ability to form active complexes with a polypeptide of the other aspect of the invention (the ability of the polypeptide to undergo complex formation may be affected or the activity of the resulting complex may be affected); and (iii) the activity of the complex. Methods for determining anticoagulant activity are discussed above and also in the Examples section below. Such methods include the prothrombin test. Agonist or antagonist activity may also be assayed for by using the assay described herein for assessing inhibition of the extrinsic tenase complex.

Methods for determining if the test compound enhances or decreases the activity of a polypeptide or polypeptide complex of the invention will be known to persons skilled in the art and include, for example, docking experiments/software or X-ray crystallography.

The polypeptide or polypeptide complex of the invention that is employed in the screening methods of the invention may be free in solution, affixed to a solid support, borne on a cell surface or located intracellularly.

Test compounds (i.e. potential modulators) may come in various forms, including natural or modified substrates, enzymes, receptors, small organic molecules such as small natural or synthetic organic molecules of up to 2000Da, preferably 800Da or less, peptidomimetics, inorganic molecules, peptides, polypeptides, antibodies, structural or functional mimetics of the aforementioned.

Test compounds may be isolated from, for example, cells, cell-free preparations, chemical libraries or natural product mixtures. These modulators may be natural or modified substrates, ligands, enzymes, receptors or structural or functional mimetics. For a suitable review of such screening techniques, see Coligan et al., Current Protocols in immunology 1(2):Chapter 5 (1991).

Compounds that are most likely to be good antagonists, agonistsor agonists and antagonists are molecules that bind to the polypeptide or polypeptide complex of the invention.

Modulators (e.g. antagonists) may alternatively function by virtue of competitive binding to a receptor for a polypeptide or polypeptide complex of the invention.

Modulators (e.g. agonists) may alternatively function by binding to a receptor for a polypeptide or polypeptide complex of the invention and increasing the affinity of the binding between the receptor and the polypeptide or polypeptide complex of the invention.

Potential modulators (e.g. antagonists) include small organic molecules, peptides, polypeptides and antibodies that bind to the polypeptide of the invention and thereby inhibit or extinguish its activity. In this fashion, binding of the polypeptide or polypeptide complex to normal cellular binding molecules may be inhibited, such that the natural biological activity of the polypeptide or polypeptide complex is prevented.

In certain of the embodiments described above, simple binding assays may be used, in which the adherence of a test compound to a surface bearing the polypeptide or polypeptide complex is detected by means of a label directly or indirectly associated with the test compound or in an assay involving competition with a labelled competitor.

Another technique for drug screening which may be used provides for high throughput screening of compounds having suitable binding affinity to the polypeptide or polypeptide complex of interest (see International patent application WO84/03564). In this method, large numbers of different small test compounds are synthesised on a solid substrate, which may then be reacted with the polypeptide or polypeptide complex of the invention and washed. One way of immobilising the polypeptide or polypeptide complex is to use non-neutralising antibodies. Bound polypeptide or polypeptide complex may then be detected using methods that are well known in the art. Purified polypeptide or polypeptide complex can also be coated directly onto plates for use in the aforementioned drug screening techniques.

In silico methods may also be used to identify modulators. The activity of the modulators may then be confirmed, if desired, experimentally.

A fifteenth aspect of the invention provides a pharmaceutical composition comprising a polypeptide of the first or second aspect of the invention, a nucleic acid molecule of the third aspect of the invention, a vector of the fourth aspect of the invention, a host cell of the fifth aspect of the invention, a complex of the ninth aspect of the invention, an antibody of the eleventh aspect of the invention, an antivenom of the thirteenth aspect of the invention, a modulator identified by the method of the fourteenth aspect of the invention.

In one embodiment, the pharmaceutical composition contains a polypeptide of the first aspect of the invention or a nucleic acid molecule encoding the same.

In one embodiment, the pharmaceutical composition contains a polypeptide of the second aspect of the invention or a nucleic acid molecule encoding the same.

In one embodiment, the pharmaceutical composition comprises: (i) a polypeptide of the first aspect of the invention or a nucleic acid molecule encoding the same; and (ii) a polypeptide of the second aspect of the invention or a nucleic acid molecule encoding the same.

Where the pharmaceutical composition comprises a polypeptide of the first aspect of the invention and a polypeptide of the second aspect of the invention the polypeptides may be provided in the form of a complex comprising the two polypeptides or the polypeptides may be provided in the form of uncomplexed polypeptides.

In one embodiment, the ratio of the polypeptide of the first aspect of the invention with the polypeptide of the second aspect of the invention is in the range of 1:2 to 2:1; more preferably in the range of 1:1.5 to 1.5:1; more preferably 1:1.25 to 1.25:1; more preferably 1:1.15 to 1.15:1; more preferably 1:1.1 to 1.1:1; more preferably 1:1.05 to 1.05:1; and yet more preferably about 1:1. Where the polypeptides are present in a ratio of 1:1, the polypeptides are suitably present as atetramer, i.e. the complex comprises 2 polypeptides of the first aspect of the invention and 2 polypeptides of the second aspect.

The pharmaceutical compositions of the present invention may comprise a pharmaceutically acceptable carrier. The compositions may be administered alone or in combination with at least one other agent, such as a stabilizing compound, which may be administered in any sterile, biocompatible pharmaceutical carrier including, but not limited to, saline, buffered saline, dextrose, and water.

The pharmaceutical compositions utilized in this invention may be administered by any number of routes including, but not limited to, oral, intravenous, intramuscular, intra-arterial, intracerebroventricularly, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they can be placed in an appropriate container and labeled for treatment of an indicated condition. Such labeling may include the amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating the ED50 (the dose therapeutically effective in 50% of the population) or LD50 (the dose lethal to 50% of the population) statistics. The dose ratio of toxic to therapeutic effects is the therapeutic index, and it can be expressed as the LD50/ED50 ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED50 with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Whilst the above discussion is said to be in relation to the pharmaceutical compositions of the invention, it will be appreciated that the discussion may pertain also to the other medical products or aspects of the invention including the “combined preparations” and “medicaments” of the invention.

A sixteenth aspect of the invention provides a polypeptide of the first or second aspect of the invention, a nucleic acid molecule of the third aspect of the invention, a vector of the fourth aspect of the invention, a host cell of the fifth aspect of the invention, a complex of the ninth aspect of the invention, an antibody of the eleventh aspect of the invention, an antivenom of the thirteenth aspect of the invention, a modulator identified by the method of the fourteenth aspect of the invention for use in medicine. In one embodiment, the medical use is for treating a patient in need of anticoagulant therapy.

By “a patient in need of anticoagulant therapy” we include patients suffering from, or susceptible to, a condition with which excessive blood clotting is associated. Excessive blood clotting is any degree of clotting that, for the particular patient, may be detrimental to the health of a patient. Such conditions may include one or more of the following: a thromboembolic disease, cerebral thrombosis, coronary arterial disease, myocardial infarction, cerebral vascular disease, stroke, pulmonary embolism, venous thrombosis, deep vein thrombosis, phlebitis, superficial, peripheral arterial disease, disseminated intravascular coagulation (DIC), thrombophlebitis, phlebothrombosis, restenosis, peripheral anginaphraxis, angiopathic thrombosis, ischemic cerebral vascular thrombosis, thrombosis related disease, unstable angina, unstable stenocardia, and thromboangitis obliterans.

As used herein, the term “treatment” (and grammatical variants thereof) refers to any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment. A seventeenth aspect of the invention provides a combined preparation for treating a patient in need of anticoagulant therapy, the combined preparation comprising:
(i) a polypeptide according to the first aspect of the invention or a nucleic acid molecule encoding the same; and
(ii) a polypeptide according to the second aspect of the invention or a nucleic acid molecule encoding the same.

(i) and (ii) may be provided in the form of a complex of the ninth aspect of the invention or separately.

An eighteenth aspect of the invention provides for the use of a polypeptide of the first or second aspect of the invention, a nucleic acid molecule of the third aspect of the invention, a vector of the fourth aspect of the invention, a host cell of the fifth aspect of the invention or a complex of the ninth aspect of the invention in the manufacture of a medicament for use in treating a patient in need of anticoagulant therapy.

In one embodiment of the eighteenth aspect of the invention there is provided the use of a polypeptide of the first aspect of the invention or a nucleic acid molecule encoding the same in the manufacture of a medicament for use in treating a patient in need of anticoagulant therapy.

Optionally, the medicament is simultaneously, separately or sequentially administered with a polypeptide of the second aspect of the invention or a nucleic acid molecule encoding the same.

In one embodiment, the patient has already been administered a polypeptide of the second aspect of the invention or a nucleic acid molecule encoding the same.

In one embodiment of the eighteenth aspect of the invention there is provided the use of a polypeptide of the second aspect of the invention or a nucleic acid molecule encoding the same in the manufacture of a medicament for use in treating a patient in need of anticoagulant therapy.

Optionally, the medicament is simultaneously, separately or sequentially administered with a polypeptide of the first aspect of the invention or a nucleic acid molecule encoding the same.

In one embodiment, the patient has already been administered a polypeptide of the first aspect of the invention or a nucleic acid molecule encoding the same.

A nineteenth aspect of the invention provides for the use of:
(i) a polypeptide according to the first aspect of the invention or a nucleic acid molecule encoding the same; and
(ii) a polypeptide according to the second aspect of the invention or a nucleic acid molecule encoding the same
   in the manufacture of a combined preparation for treating a patient in need of anticoagulant therapy.

By a “combined preparation” as used herein we include pharmaceutical preparations which include: (i) a polypeptide according to the first aspect of the invention or a nucleic acid molecule encoding the same; and (ii) a polypeptide according to the second aspect of the invention or a nucleic acid molecule encoding the same. Components (i) and (ii) may be present in a single formulation or may be present as separate formulations. Where components (i) and (ii) are in a single formulation they may be provided in the form of a complex or in the form of uncomplexed polypeptides (or of course a mixture of both).

Thus, the active ingredients may be administered at the same time (e.g. simultaneously) or at different times (e.g. sequentially) and over different periods of time, which may be separate from one another or overlapping.

If there is separate or sequential administration, the delay in administering the second therapeutic agent should not be such as to lose the benefit of a synergistic therapeutic effects of the pharmaceutical combination of the therapeutic agents as achieved according to the present invention. The time delay between administration of the components will vary depending on the exact nature of the components, the interaction there between, and their respective half-lives.

The combination partners may be administered in any order.

In one embodiment, the ratio of component (i) to component (ii) is in the range of 1:2 to 2:1; more preferably in the range of 1:1.5 to 1.5:1; more preferably 1:1.25 to 1.25:1; more preferably 1:1.15 to 1.15:1; more preferably 1:1.1 to 1.1:1; more preferably 1:1.05 to 1.05:1; and yet more preferably about 1:1.

A twentieth aspect of the invention provides a method of treating a patient in need of anticoagulant therapy, the method comprising administering to the patient a polypeptide of the first or second aspect of the invention, a nucleic acid molecule of the third aspect of the invention, a vector of the fourth aspect of the invention, a host cell of the fifth aspect of the invention, a complex of the ninth aspect of the invention or a pharmaceutical composition of the fifteenth aspect of the invention.

A twenty-first aspect of the invention provides a method of treating a patient in need of anticoagulant therapy, the method comprising administering to the patient:
(i) a polypeptide according to the first aspect of the invention or a nucleic acid molecule encoding the same; and
(ii) a polypeptide according to the second aspect of the invention or a nucleic acid molecule encoding the same.

As discussed above, (i) and (ii) may be present as separate formulations or as a single formulation comprising (i) and (ii). Where in the form of separate formulations, (i) and (ii) may be administered separately, sequentially or simultaneously.

(i) and (ii) may be provided in the form of a complex of the ninth aspect of the invention.

A twenty-second aspect of the present invention provides a method of treating snake-bite in a patient, the method comprising administering to the patient a polypeptide of the first or second aspect of the invention, a nucleic acid molecule of the third aspect of the invention, a vector of the fourth aspect of the invention, a host cell of the fifth aspect of the invention, a complex of the ninth aspect of the invention or a pharmaceutical composition of the fifteenth aspect of the invention. A twenty-third aspect of the present invention provides use of a polypeptide of the first or second aspect of the invention, a nucleic acid molecule of the third aspect of the invention, a vector of the fourth aspect of the invention, a host cell of the fifth aspect, a complex of the ninth aspect of the invention or a pharmaceutical composition of the fifteenth aspect of the invention in the manufacture of a medicament for treating snake-bite in a patient.

Whilst the invention has in certain places been described in relation to particular aspects of the invention, the skilled reader will appreciate that the comments may apply equally to other aspects of the invention and the description should be construed accordingly.

The practice of the present invention will employ, unless otherwise indicated, conventional techniques of molecular biology, microbiology, and recombinant DNA technology which are within the skill of those working in the art. Such techniques are explained fully in the literature. Examples of texts for consultation include the following: Sambrook Molecular Cloning; A Laboratory Manual, Third Edition (2000) and subsequent editions.

### EXAMPLES

Reported herein are the purification and characterization of a three-finger toxin that mediates anticoagulant activity from the venom of an elapid snake H. haemachatus (African Ringhals cobra). Although it has mild anticoagulant activity, its synergistic interaction with the second three-finger toxin enhances its anticoagulant effects. Described herein is the characterization of the complex formation. The anticoagulant protein and its complex specifically inhibit the activation of FX by TF-FVIIa complex. This is the first unique synergistic complex between three-finger toxins known to exhibit anticoagulant effects by the inhibition of the TF-FVIIa complex.

### Materials and Methods

Materials—Lyophilized H. haemachatus venom was obtained from African Reptiles and Venoms, Gauteng, South Africa. Thromboplastin with calcium (for prothrombin time assays), Russell's viper venom (RVV) (for Stypven time assays), thrombin reagent (for thrombin time assays), benzamidine hydrochloride and 4-vinylpyridine were purchased from Sigma (St. Louis, Mo., USA). β-mercaptoethanol was purchased from Nacalai Tesque (Kyoto, Japan). The chromogenic substrates H-D-Ile-Pro-Arg-p-nitroanilide (pNA) dihydrochloride (2HCl), (S-2288), pyro-Glu-Pro-Arg-pNA.HCl (S-2366), H-D-Phe-Pip-Arg-pNA.2HCl (S-2238), H-D-Pro-Phe-Arg-pNA.2HCl (S-2302), Z-D-Arg-Gly-Arg-pNA.2HCl (S-2765), pyro-Glu-Gly-Arg-pNA.HCl (S-2444), benzoyl-Ile-Glu(GluγOMe)-Gly-Arg-pNA.HCl (S-2222), H-D-Val-Leu-Lys-pNA.2HCl (S-2251), H-D-Val-Leu-Arg-pNA.2HCl (S-2266) and MeO-Suc-Arg-Pro-Tyr-pNA.HCl (S-2586) were from Chromogenix AB, Stockholm. Spectrozyme®FIXa (H-D-Leu-Ph′Gly-Arg-pNA.2AcOH) was obtained from American Dignostica Inc., Stamford, Conn. All substrates were reconstituted in deionized water prior to use. Freeze dried recombinant human tissue factor (Inovin) was purchased from Dade Behring Marburg, Germany. Human plasma was donated by healthy volunteers. All other chemicals and reagents used were of highest purity available.

Purification of anticoagulant protein—H. haemachatus crude venom (100 mg in 1 ml distilled water) was applied to a Superdex 30 gel filtration column (1.6×60 cm) equilibrated with 50 mM Tris-HCl buffer (pH 7.4) and eluted using the same buffer, using a ÄKTA Purifier system (Amersham Biosciences, Uppsala, Sweden). Individual fractions were assayed for anticoagulant activity using the prothrombin time coagulation test (see below). Fractions with potent anticoagulant activity were pooled and sub-fractionated on a cation exchange column, using the same chromatographic system. The anticoagulant fraction was loaded on to a Uno S-6 (Bio-Rad, Hercules, Calif.; column volume, 6 ml) column equilibrated with 50 mM Tris-HCl buffer, pH 7.5. Bound proteins were eluted with a linear gradient of 1 M NaCl in the same buffer. Fractions collected were assayed for anticoagulant activity. The anticoagulant peaks obtained from cation-exchange chromatography were applied to a Jupiter C18 (1×25 cm) column equilibrated with 0.1% trifluoroacetic acid (TFA). The bound proteins were eluted using a linear gradient of 80% acetonitrile (ACN) in 0.1% TFA. Individual peaks were collected, lyophilized and examined for anticoagulant activity and subsequently rechromatographed on a narrow bore Pepmap column using a Chromeleon micro-liquid chromatography system (LC Packings, San Francisco, Calif.).

Electrospray ionization mass spectrometry (ESI-MS)—The homogeneity and mass of the anticoagulant proteins were determined using ESI-MS using a Perkin-Elmer Sciex API-300 LC/MS/MS system. Typically, RP-HPLC fractions were directly used for analysis. Ionspray, orifice and ring voltages were set at 4600, 50 and 350 V, respectively. Nitrogen was used as a nebulizer and curtain gas. An LC-10AD Shimazdu pump was used for solvent delivery (40% ACN in 0.1% TFA) at a flow rate of 50 μl/min. BioMultiview software (Perkin-Elmer Sciex) was used to analyze and deconvolute raw mass spectra.

Reduction and pyridylethylation—Purified proteins were reduced and pyridylethylated using procedures as described earlier (24). Briefly, proteins (0.5 mg) were dissolved in 500 μl of denaturant buffer (6 M guanidium hydrochloride, 0.25 M Tris-HCl, 1 mM EDTA, pH 8.5). After adding of 10 μl of β-mercaptoethanol, the mixture was incubated under vacuum for 2 h at 37° C. 4-vinylpyridine (50 μl) was added to the mixture and kept at room temperature for 2 h. Pyridylethylated proteins were purified on an analytical Jupiter C18 column (4.6×250 mm) using a gradient of ACN in 0.1% (v/v) TFA at a flow rate of 0.5 ml/min.

N-terminal sequencing—N-terminal sequencing of the native and S-pyridylethylated proteins were performed by automated Edman degradation using a Perkin-Elmer Applied Biosystems 494 pulsed-liquid phase sequencer (Procise) with an online 785A PTH-amino acid analyzer.

Reconstitution of the anticoagulant complex—Preliminary studies indicated that the active anticoagulant protein interacted with another venom protein forming a synergistic complex. The complex was reconstituted for various in vitro experiments immediately prior to the experiment by incubating equimolar concentration of the two proteins (unless mentioned otherwise) at 37° C. for a period of 5 min in 50 mM Tris-buffer (pH 7.4).

Anticoagulant activity—The anticoagulant activities of H. haemachatus venom and its fractions was determined by four coagulation tests using a BBL fibrometer:
1. Recalcification time: The recalcification times were determined according to the method of Langdell et al. (25). 50 mM Tris-HCl buffer (pH 7.4) (100 μl), plasma (100 μl) and various concentrations of venom or its fraction (50 μl) were preincubated for 2 min at 37° C. Clotting was initiated by the addition of 50 μl of 50 mM CaCl₂.
2. Prothrombin time: The prothrombin times were measured according to the method of Quick (26). 100 μl of 50 mM Tris-HCl buffer (pH 7.4), 100 μl of plasma and 50 μl of venom or its fractions were preincubated for 2 min at 37° C. Clotting was initiated by the addition of thromboplastin with calcium reagent (150 μl) which can be purchased from Sigma (St. Louis, Mo., USA).
   For studying the role of electrostatic interactions, the anticoagulant activity of a specific concentration of hemextin A (4.4 μM), hemextin B (4.4 μM) and hemextin AB complex (0.11 μM) was monitored in 50 mM Tris-HCl (pH 7.4) containing various concentrations of NaCl (35 mM to 150 mM).
   For studying the role of hydrophobic interactions, the anticoagulant activity of a specific concentration of hemextin A (4.4 μM), hemextin B (4.4 μM) and hemextin AB complex (0.11 μM) was monitored in 50 mM Tris-HCl (pH 7.4) containing various concentrations of glycerol (125 mM to 250 mM).
2. Stypven time: Stypven time measurements were determined according to the method of Hougie (27). Plasma (100 μl), 50 mM Tris-HCl buffer (pH 7.4) (100 μl) and RVV (0.01 μg in 100 μl) and individual proteins or the reconstituted complex (50 μl) were preincubated for 2 min at 37° C. Clotting was initiated by the addition of 50 mM CaCl₂ (50 μl).
3. Thrombin time: Thrombin time was determined according to the method of Jim (28). Individual proteins or the reconstituted complex were incubated with 100 μl of plasma and 100 μl of 50 mM Tris-HCl buffer (pH 7.4) for 2 min at 37° C. in a total volume 250 μl. Clotting was initiated by the addition of standard thrombin reagent (0.01 NIH units in 50 μl).

Gel filtration chromatography—The complex formation between anticoagulant proteins was examined by gel filtration chromatography using a Superdex 30 gel filtration column (1.6×60 cm) using ÄKTA Purifier. The column was equilibrated with 50 mM Tris-HCl buffer (pH 7.4) at a flow rate of 1 ml/min. Individual proteins and equimolar mixture of anticoagulant proteins (incubated for a period of 30 minutes at 37° C.) were loaded on to the column and eluted in the same buffer. Elution was followed at 280 nm.

Purification of FVIIa—Large scale preparation of FVIIa was carried out in the way as described in (29). Briefly, 4.5 grams of FVII was purified and nanofiltered from 15000 litres of human plasma. After complete activation of FVII to FVIIa by the incubation of FVII for 18 h at 110° C., FVIIa preparation was dialysed against 20 mM citrate, pH 6.9, containing 240 mM NaCl and 13 mM glycine. The dialysed FVIIa was frozen and stored at −60° C.

Preparation of sTF—Recombinant Human sTF (TF Minus the Trans-Membrane and the intracellular domain and containing amino acids 1-219) was prepared as described (30). Briefly, the expression vector for the production of sTF was constructed and expressed in Saccharomyces cerevisae. The recombinant sTF was secreted into the culture broth and isolated by a two step column chromatographic procedure.

Reconstitution of the extrinsic tenase complex—TF-FVIIa complex was reconstituted by incubating 10 pM FVIIa with 70 nM of recombinant human TF (Innovin) in Buffer A (20 mM HEPES, 150 mM NaCl, 10 mM CaCl₂ and 1% BSA, pH 7.4) for 10 min at 37° C. Then FX was added to the mixture to obtain a final concentration of 30 nM. The activation was stopped by the addition 50 μl of stop buffer (20 mM HEPES, 150 mM NaCl, 50 mM EDTA and 1% BSA, pH 7.4) to 50 μl aliquots of the reaction mixture after 15 min incubation. FXa formed was measured by the hydrolysis 1 mM of S-2222 in Buffer A in a microtiter plate reader at 405 nm. The inhibitory effect on extrinsic tenase activity was determined by adding the individual proteins or the anticoagulant complex 15 min prior to FX addition.

Serine protease specificity—The selectivity profile of anticoagulant proteins and their complex was examined against 12 serine proteases—procoagulant serine proteases (FIXa, FXa, FXIa, FXIIa, plasma kallikrein and thrombin), anticoagulant serine protease (APC), fibrinolytic serine proteases (urokinase, t-PA and plasmin) and classical serine proteases (trypsin and chymotrypsin). Various concentrations of purified hemextin A/hemextin B and reconstituted hemextin AB complex were preincubated with each of the enzymes (Table 1) for a period of five minutes at a temperature of 37° C., followed by the addition of appropriate chromogenic substrate.

TABLE 1

| Serine | | Control | Observed |
|---|---|---|---|
| protease | Substrates | Inhibitor | Effect |
| FVIIa | S-2288 | Benzamidine | Inhibition |
| FVIIa-sTF | S-2288 | Benzamidine | Inhibition |
| FVIIa-TF | S-2288 | Benzamidine | Inhibition |
| Factor IXa | Spectrozyme fIXa | Benzamidine | No inhibition |
| FXa | S-2765 | Benzamidine | No inhibition |
| Factor XIa | S-2266/S-2302/S-2366 | Benzamidine | No inhibition |
| Factor XIIa | S-2302 | Benzamidine | No inhibition |
| Plasma | S-2266/S-2302/S-2288 | Benzamidine | Inhibition |
| Kallikrein | | | |
| Thrombin | S-2238 | Benzamidine | No inhibition |
| t-PA | S-2288 | Benzamidine | No inhibition |
| APC | S-2366 | Benzamidine | No inhibition |
| Urokinase | S-2444/S-2484 | Benzamidine | No inhibition |
| Plasmin | S-2251 | Aprotinin | No inhibition |
| Chymotrypsin | S-2586 | Aprotinin | No inhibition |
| Trypsin | S-2222 | Benzamidine | No inhibition |

For studies with FXIa, kallikrein and urokinase, the appropriate substrates were determined prior to their screening against the inhibitors. For FXIa, the Vₘₐₓ for the amidolytic activity corresponding to the chromogenic substrates S-2266, S-2302 and S-2366 was determined. S-2302 was the substrate with the highest Vₘₐₓ and thus was used in the screening studies. Similar studies with kallikrein and urokinase were carried out with substrates S-2266, S-2302 and S-2288 for kallikrein and S-2444 and S-2484 for urokinase. In a total volume of 200 μl in the individual wells of the microtiter plate, final concentrations of FVIIa (300 nM)/S-2288, FVIIa-sTF (30 nM)/S-2288, FXa (0.75 nM)/S-2765, α-thrombin (0.66 nM)/S-2238, plasmin (2 nM)/S-2366, FIXa (3 μM)/spectrozyme®fIXa, FXIa (0.34 nM)/S-2366, FXIIa (0.4 nM)/S-2302, recombinant tissue plasminogen activator (80 nM)/S-2288, activated protein C (0.34 nM)/S-2366, urokinase/S-2444, plasma kallikrein (0.4 nM)/S-2302, trypsin (2.17 nM)/S-2222 and chymotrypsin (0.4 nM)/S-2586 were measured. The kinetic rate of substrate hydrolysis (mOD/min) was measured over 5 min.

Determination of kinetic constants for substrate hydrolysis—All studies were in assay buffer containing 50 mM Tris-HCl, pH 8.0, containing 150 mM NaCl, 10 mM CaCl₂, and 1% BSA at 37° C. The kinetics of hydrolysis of the chromogenic substrate S-2288 by FVIIa-sTF was measured prior to examining the inhibitory effects of individual hemextins and the hemextin AB complex. Reactions were initiated by the addition of S-2288 (0-5 mM) to the individual wells of a 96-well plate containing FVIIa (30 nM) in complex with sTF (100 nM) in a final volume of 180 μl. Initial reaction velocities were measured as a linear increase in the absorbance at 405 nm (A₄₀₅ nm) over 5 min, with a SPECTRAmax Plus® temperature-controlled microplate spectrophotometer (Molecular Devices, Sunnyvale, Calif.). The Kₘ was derived from the nonlinear regression fit of the determined velocities, the value of which was 2.79 mM.

Kinetics of inhibition—The inhibitory potency of anticoagulant complex was measured over a range of substrate concentrations. Reactions were initiated by the addition of S-2288 to premixed enzyme-cofactor and inhibitor in the wells of a microtiter plate. Reactions with FVIIa-sTF contained 0.0125-0.05 μM of inhibitor complex and 0 to 3 mM of S-2288. The initial velocities were measured over 5 min under steady-state conditions and were fit by reiterative nonlinear regression to Equation 1, describing a classical non-competitive inhibitor, to derive the Kᵢ value.

V=V_{max [S]/(}1+[I]Kᵢ)/{Kₘ+[S]} (Eq. 1)

Isothermal titration calorimetry (ITC) studies—The interaction of anticoagulant hemextin AB complex with FVIIa was monitored with a VP-ITC titration calorimetric system (Microcal Inc., Northampton, Mass.). The instrument was calibrated using the built-in electrical calibration check. FVIIa (10 μM) in 50 mM Tris-HCl buffer and 10 mM CaCl₂ (pH 7.4) in the calorimetric cell was titrated with reconstituted anticoagulant complex (0.2 mM) dissolved in the same buffer in a 250 μl injection syringe, with continual stirring at 300 rpm at 37° C. All the protein solutions were filtered and degassed prior to titration. The first injections presented defects in the baseline and these data were not included in the fitting process. The calorimetric data were processed and fitted to the single set of identical sites model using Microcal Origin Version 7.0 data analysis software supplied with the instrument. The total heat content Q of the solution (determined relative to zero for the unliganded species) contained in the active cell volume, Vₒ, was calculated according to the following Equation 2, where K is the binding affinity constant, n is the number of sites, ΔH is the enthalpy of ligand binding, Mₜ and Xₜ is the bulk concentration of macromolecule and ligand, respectively, for the binding X+M [symbol] XM

$$Q = \frac{{nM}_{t } Δ {HV}_{0}}{2} [ 1 + \frac{{X}_{t}}{{nM}_{t}} + \frac{1}{{nK}_{a} {M}_{t}} - \sqrt{{( 1 + \frac{{X}_{t}}{{nM}_{t}} + \frac{1}{{nK}_{a} {M}_{t}} )}^{2} - \frac{4 {X}_{t}}{{nM}_{t}}} ] \qquad ( Eq . 2 )$$

The change in heat (ΔQ) measured between the completions of two consecutive injections is corrected for dilution of the protein and ligand in the cell according to standard Marquardt methods. The free energy change (AG) during the interaction was calculated by using the relationship: ΔG=ΔH−TΔS=RT ln Kₐ, where T is the absolute temperature and R is the universal gas constant.

CD spectroscopic studies—Far UV CD spectra (260-190 nm) were recorded using a Jasco J-810 spectropolarimeter (Jasco Corporation, Tokyo, Japan). All measurements were carried out at room temperature using 0.1 cm pathlength stoppered cuvettes. The instrument optics was flushed with 30 l/min of nitrogen gas. The spectra were recorded using a scan speed of 50 nm/min, resolution 0.2 nm, and band width 2 nm. For each spectrum, a total of 6 scans were recorded, averaged and baseline subtracted. Conformation of hemextin A and hemextin B at different concentrations were monitored in 50 mM Tris-HCl buffer (pH 7.4). To study the complex formation, titration experiments were carried out by keeping the concentration of the hemextin A constant at 0.5 mM, and varying the concentrations of hemextin B.

Determination of molecular diameters—The diameter of the hemextin AB complex and the individual hemextins were determined in both the gas and solution phases.

(A) Gas Phase Electrophoretic Mobility Macromolecule Analyzer (GEMMA)—The molecular diameters in the gas phase were determined with GEMMA (71, 72) using a nano-differential mobility analyzer, model 3980 (TSI, St Paul, Minn., USA), and a standard CPC type 3025 (TSI, St Paul, Minn., USA). The instrument was operated in the ‘cone jet’ mode with an operating voltage between 2.5 and 3.0 kV, resulting in currents from 200 to 300 nA. Filtered ambient air at 2 l/min and a concentric sheath gas flow of filtered CO₂ at 0.1/min was used to stabilize the electrospray against corona discharge. Sample solutions of hemextin A (4 ng/ml) and hemextin B (4 ng/ml) were prepared in 20 mM ammonium acetate (pH 7.4) immediately prior to the experiment. Hemextin AB complex (4.5 ng/ml) was reconstituted in the above buffer and was incubated at 37° C. for 10 min. Another three-finger protein, toxin C isolated and purified from the same venom was used as a control in the GEMMA experiments. The samples were infused into the electrospray chamber with an inlet flow rate of 100 nl/min. Twenty scans over the whole EM diameter range (0 to 25 nm) were recorded and averaged to obtain a GEMMA spectrum. No smoothing algorithm was applied for the data presentation.

(B) Dynamic Light Scattering (DLS)—Complex formation studies with DLS were carried out at 25° C. using a BI200SM instrument (Brookhaven Instruments Corporation, Holstvile, N.Y., USA). A vertically polarized argon ion laser (514.2 nm, 75 mW; NEC model GLG-3112) was used as the light source. Sample solutions of hemextin A (4 mM), hemextin B (4.1 mM) and hemextin AB complex (2.3 mM) were prepared immediately prior to the experiment. The hydrodynamic diameter for the hemextin AB complex and the individual hemextins were recorded at 25° C. in solutions of different ionic strengths and at different glycerol concentrations. The ionic strengths were varied by the addition of NaCl. From the measured translational diffusion coefficient (D_{T}), the hydrodynamic radius (R_{H}) can be calculated using the Stokes-Einstein relation:

D_{T}=k_{B}T/6πηR_{H} (Eq. 3),

where k_{B} is the Boltzmann constant, T is the temperature in Kelvin and η being the viscosity of the solvent. The intensity-intensity time correlation functions were obtained with a BI-9000 digital correlator equipped with the instrument. The particle size and size distribution were obtained by analyzing the field correlation function |g⁽¹⁾(τ)| using constrained regularized CONTIN method (73).

Effects of protonation—To study the effects of protonation on complex formation, additional calorimetric experiments were performed in PBS, pH 7.4, or in 10 mM MOPS, pH 7.4.

Role of electrostatic interactions—The role of electrostatic interactions in the complex formation was evaluated by performing ITC experiments in 50 mM Tris-HCl buffer of various ionic strengths. The ionic strengths of the buffers were altered by adding sodium chloride (NaCl) (35 mM to 150 mM).

Role of hydrophobic interactions—To study the role of hydrophobic interactions in the complex formation, experiments were performed in 50 mM Tris-HCl buffer (pH 7.4) containing various concentrations of glycerol (125 mM to 250 mM).

Size exclusion chromatography (SEC) studies—All SEC experiments were carried out at room temperature on a pre-packed Superdex 75 gel filtration column (1.6×60 cm) using a ÄKTA Purifier system (Amersham Biosciences, Uppsala, Sweden). The column was eluted with 50 mM Tris-HCl buffer (pH 7.4) or the specified elution buffer, at a flow rate of 1 ml/min. The sample volume applied to the column was 4 ml. The column was calibrated using ovomucoid (28 kD) ribonuclease (15.6 KD), cytochrome C (12 KD), apoprotinin (7 KD) and pelovaterin (4 KD) (20) as molecular weight markers. The void volume was determined by running Blue Dextran. The column was equilibrated with at least two bed volumes of the elution buffer prior to each run. Electrostatic contributions in the hemextin AB complex formation were studied by monitoring its elution in 50 mM Tris-HCl buffer (pH 7.4) of different concentrations of NaCl (75 mM and 150 mM). Hydrophobic contributions for the complex formation were determined by recording its elution in 50 mM Tris-HCl buffer (pH 7.4). In both studies, the column was first equilibrated with the desired buffer prior to the application of the reconstituted hemextin AB complex in the respective buffer to the column. Elution of protein was monitored by absorbance at 280 nm.

1D-NMR spectroscopic studies—One-dimensional proton NMR experiments were carried out using Bruker 600 MHz, equipped with a modern cryo-probe, and electronic variable temperature unit. The spectra were acquired using Topspin software (Bruker) interfaced to the spectrometer. Hemextin A (0.5 mM) and hemextin B (0.5 mM) were prepared in 50 mM Tris-HCl buffer (pH 7) and transferred to a 5 mm Willmad NMR tube. All deuterated solvents were purchased from Aldrich Laboratories with 99.9% isotopic purity. The spectral width was set to 16 p.p.m. for experiments in ¹H₂O and the transmitter/carrier was positioned on the water signal to minimize any artifacts. The large resonance due to the water protons was suppressed by the WATERGATE pulse sequence. Typically, 128 scans were averaged for each FID before apodization and then performing the Fourier transformation. ¹H chemical shifts were referenced to a sodium 2,2-dimethyl-2-silapentane-5-sulfonate solution (DSS).

### Results

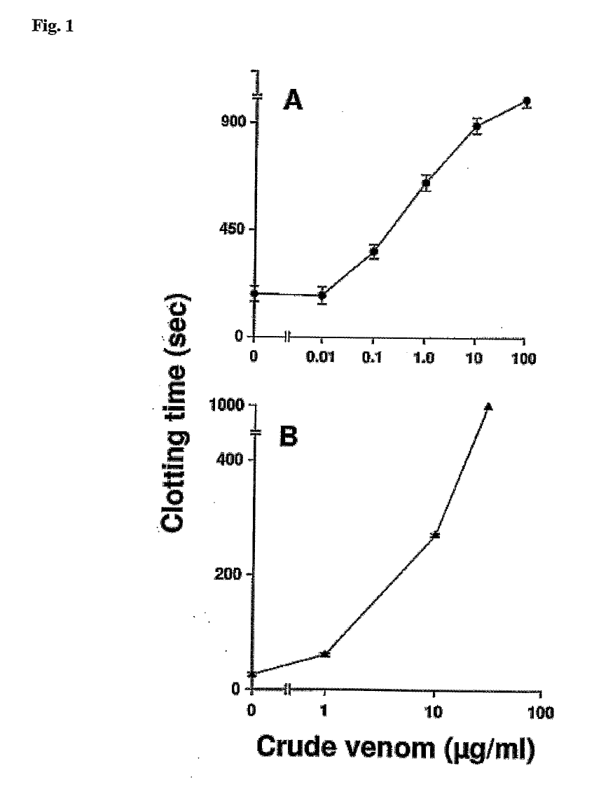

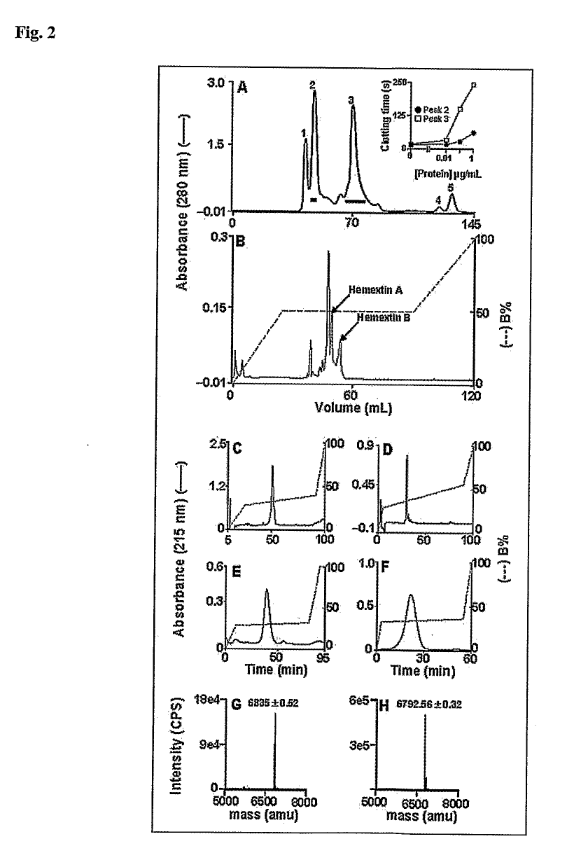

Purification of the anticoagulant protein—Crude venom of H. haemachatus exhibited potent anticoagulant activity in both recalcification and prothrombin time assays (FIGS. 1A and B). To purify the anticoagulant protein, the crude venom was size fractionated by gel filtration chromatography (FIG. 2A). Fractions corresponding to peaks 2 and 3 contained anticoagulant proteins as determined by prothrombin time assays. Peak 2 corresponded to proteins, mostly containing PLA₂ that have been characterized earlier (31). This peak, however, had milder anticoagulant activity compared to peak 3 (inset FIG. 2A). Accordingly, the focus was on isolating the anticoagulant protein from peak 3, which was fractionated further using cation exchange chromatography on Uno S column (FIG. 2B). Only peak A exhibited mild anticoagulant activity. During preliminary studies, it was found that the anticoagulant activity of peak A was potentiated by peak B (see below). Since this anticoagulant complex specifically inhibited the extrinsic tenase complex (described below), it was named hemextin (Hemachatus extrinsic tenase inhibitor) and the individual proteins hemextin A and B, respectively. Fractions corresponding to both hemextins A and B were pooled separately and purified using RP-HPLC (FIGS. 2C and D) and capillary liquid chromatography (FIGS. 2E and F). The homogeneity and mass of the individual proteins were determined by ESI-MS. Mass spectra of hemextins A and B showed three peaks of mass/charge ratios ranging from three to six charges (data not shown) and their calculated molecular mass as 6835.00±0.52 and 6792.56±0.32 daltons, respectively (FIGS. 2G and H).

N-terminal sequence determination—The sequence of the first 37 amino acid residues of hemextins A and B was determined using Edman degradation (FIG. 3). The location of the cysteine residues in the proteins were confirmed by sequencing the pyridylethylated proteins. Both proteins show similarity to cardiotoxins, postsynaptic neurotoxins, fasciculin and other members of the three-toxin family (FIG. 3), and thus belong to this family of snake venom proteins.

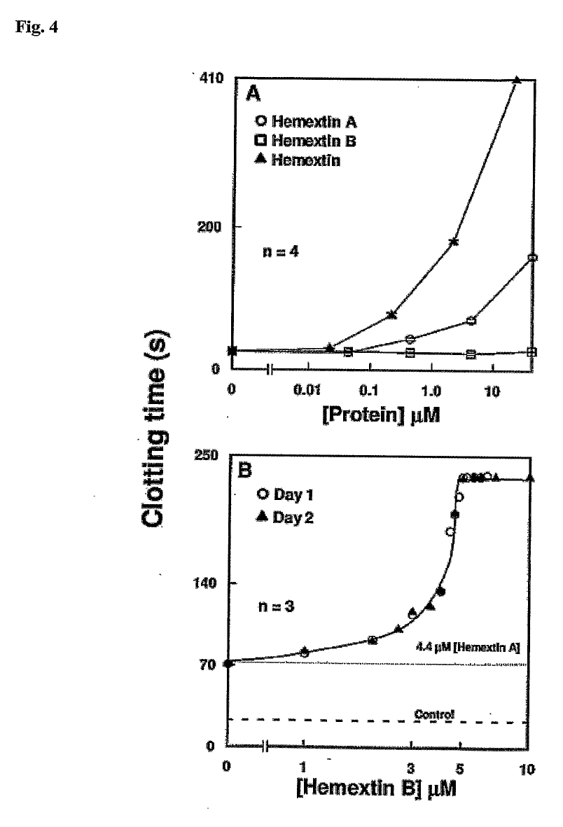

Anticoagulant activity of hemextins—The anticoagulant activity of hemextins A and B was determined using prothrombin time assay (FIG. 4A). Hemextin A prolonged the clotting time and exhibited a mild anticoagulant activity, whereas hemextin B even at higher concentrations did not show any significant effect on clotting time. Interestingly, an equimolar mixture of hemextin A and B exhibited more potent anticoagulant activity indicating synergism between these proteins (FIG. 4A). Such an increase in anticoagulant effect could be due to either the inhibition of two separate steps in the coagulation cascade or due to complex formation between them. Since hemextin B by itself has no significant effect on prothrombin time, it does not inhibit a separate step; instead, it is likely that hemextins A and B form a complex.

Complex formation between hemextins A and B—To investigate the formation of complex between the two proteins, a titration experiment was employed in the prothrombin time assay. In this experiment, concentration of hemextin A was kept constant at 4.4 μM and its anticoagulant activity was monitored with increasing hemextin B concentrations (FIG. 4B). The anticoagulant activity increases with the increasing concentrations of hemextin B until the ratio reaches 1:1. Further addition did not increase the anticoagulant effect. The results indicated that hemextins A and B form a 1:1 complex and the complex formation is crucial for the potent anticoagulant activity.

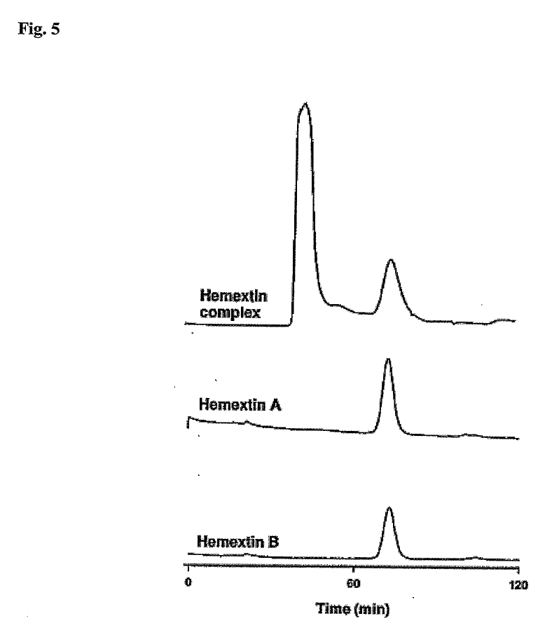

The complex formation between hemextins A and B was further confirmed using gel filtration chromatography. As shown in FIG. 5, the retention time of individual hemextins A and B was ˜70 min. However, the reconstituted complex elutes as a major peak with a retention time of ˜40 min and a minor peak with a retention time of 70 min. The appearance of the major peak with reduced retention time is consistent with the formation of complex between the two hemextins.

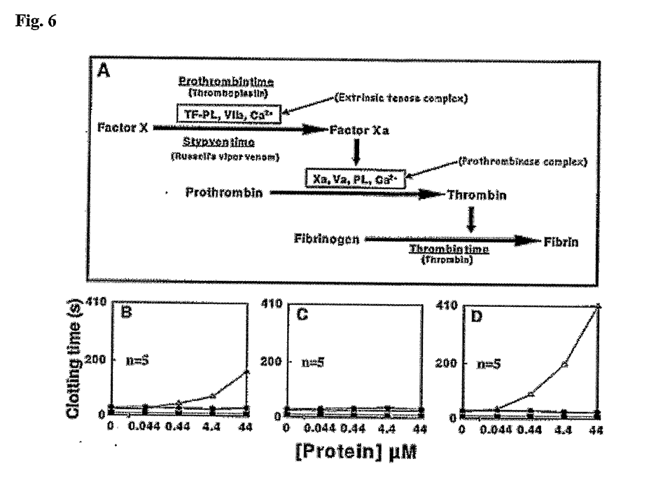

Site of anticoagulant activity—As shown earlier, hemextin A and its complex with hemextin B prolong prothrombin time (FIG. 4A). To identify the specific stage in the extrinsic coagulation pathway, we used a simple “dissection approach” was used (32, 33). Three commonly used clotting time assays, namely prothrombin time, Stypven time and thrombin time were employed (FIG. 6A). This approach is based on the principle that initiating the cascade “upstream” from the inhibited step will result in elevated clotting times, while initiating the cascade “downstream” from the inhibited step will not affect clotting times. Thus, the anticoagulant action of the individual proteins and the complex can be localized to certain activation step(s) in the cascade (FIGS. 6B-D) (for details, see 32, 33). Hemextin A exhibited a mild anticoagulant activity by prolonging the clotting time in the prothrombin time assay, but did not prolong Stypven time and thrombin time (FIG. 6B). As expected, hemextin B did not prolong clotting times in prothrombin time, Stypven time and thrombin time assays (FIG. 6C). The hemextin AB complex exhibited a potent anticoagulant activity by prolonging the clotting time in prothrombin time assay. However, the clotting times in the other two assays were not affected (FIG. 6D). These results indicate that hemextin A and hemextin AB complex affect only the extrinsic tenase complex, but not the prothrombinase complex or conversion of fibrinogen to fibrin clot.

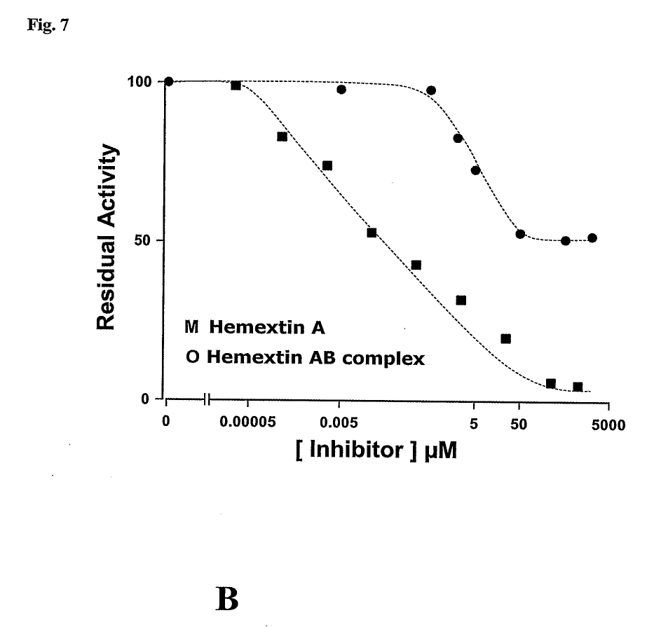

To confirm the site of inhibition, the effects of hemextins A and B and their complex on the reconstituted TF-FVIIa complex were examined (FIG. 7A). Hemextin A exhibited mild inhibitory activity at higher concentrations. Hemextin B, on the other hand, did not mediate any inhibitory activity on the enzymatic action of the extrinsic tenase complex. However, hemextin AB complex completely inhibited extrinsic tenase activity (FIG. 7A) with an IC₅₀ value (concentration of the inhibitor which inhibits 50% of the activity) of 100 nM. Neither the individual proteins nor the complex mediated any inhibitory effect on FXa amidolytic activity as observed in the later screening studies (see below). To determine the importance of hemextin AB complex formation for the inhibition of TF-FVIIa complex, a similar titration experiment was performed. The concentration of hemextin A was kept constant at 50 μM and its inhibitory activity on extrinsic tenase activity was evaluated in the presence of increasing concentrations of hemextin B. As shown in (FIG. 7B), the inhibitory activity of hemextin A increases with the increasing concentrations of hemextin B until the ratio reached 1:1. Further addition did not increase the anticoagulant effect. The results indicated that hemextins A and B form a 1:1 complex and the complex formation is crucial for the potent anticoagulant activity. No further increase in the inhibitory activity was observed after equimolar ratios of hemextins A and B. These observations further confirmed the importance of complex formation between hemextins A and B.

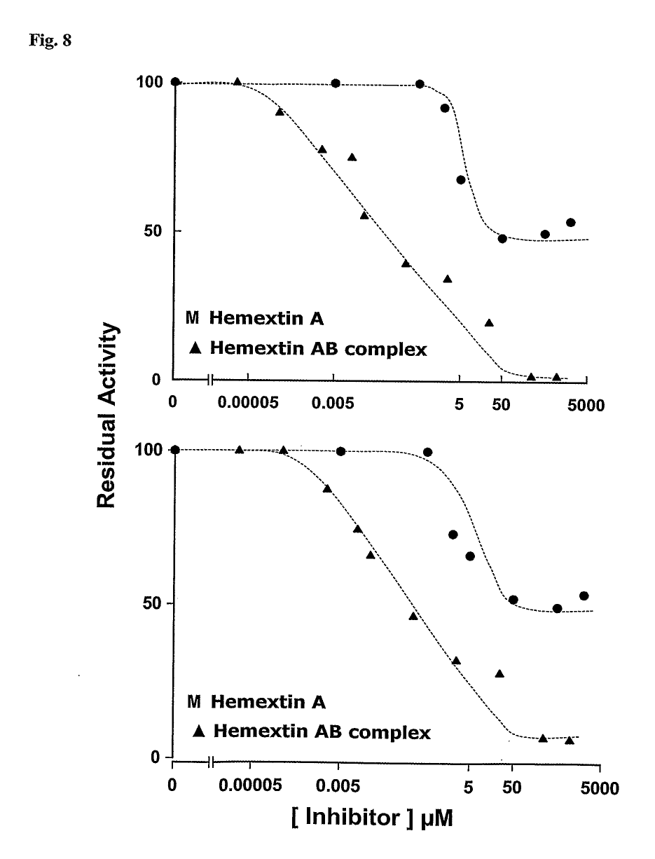

To understand the effect of phospholipids, the inhibitory activity of hemextin A and hemextin AB complex was monitored on FVIIa amidolytic activity either in the presence or absence of sTF. In both cases, potent inhibitory activity (FIGS. 8A and B) in a dose dependant manner was observed.

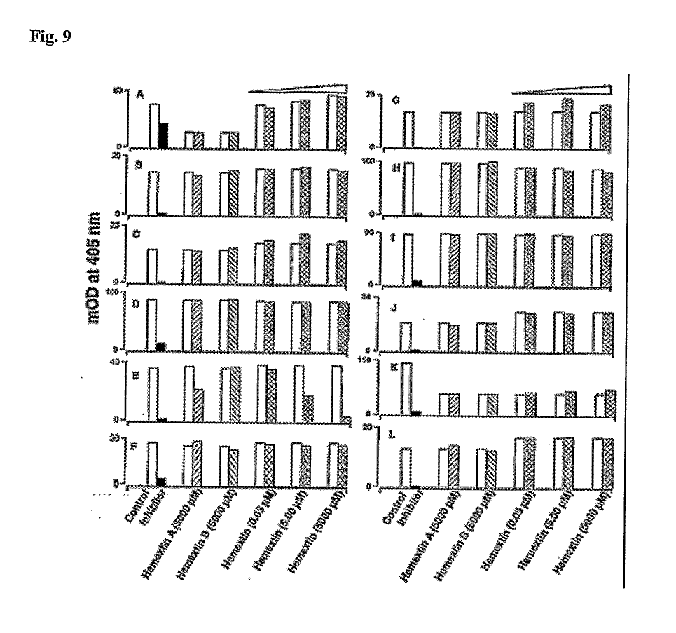

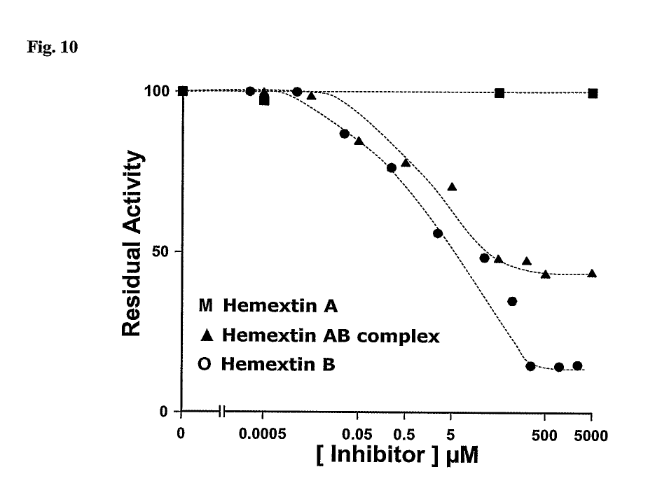

Specificity of inhibition—To determine the specificity of inhibition, hemextins A and B and their complex were screened against 12 serine proteases. As depicted in FIG. 9, no inhibitory activity was observed against any of the serine protease with the exception of FVIIa and plasma kallikrein. As with FVIIa, hemextins A and hemextin AB complex inhibit plasma kallikrein in a dose-dependant manner (FIG. 10). Hemextin B did not inhibit kallikrein's protease activity. However, the inhibitory potency towards FVIIa (either in the absence or presence of sTF) was at least 50 times higher than towards plasma kallikrein.

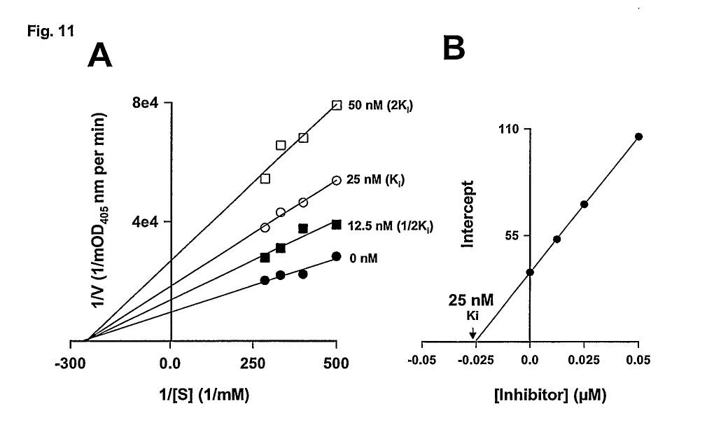

Kinetics of inhibition—To determine the mechanism of inhibition, the inhibitory kinetics of hemextin AB complex on amidolytic activity of sTF-FVIIa complex on S-2288 were examined. Kinetic studies revealed that hemextin AB complex inhibited FVIIa-sTF activity non-competitively. Lineweaver-Burk plots showed that Kₘ remained unaltered where as Vₘₐₓ decreased with increasing concentrations of inhibitor (FIG. 11A, Table 2), a characteristic of a non-competitive inhibitor. The Kᵢ value for inhibition was determined to be 25 nM (FIG. 11B). The turnover number (K_{cat}) (number of moles of substrate converted to product per mole of enzyme per min) at different concentrations of the inhibitor was also calculated. As observed in the case of classical non-competitive inhibitors, K_{cat} decreased with increasing concentrations of hemextin AB complex (Table 2). Since the amidolytic activity of FVIIa alone is very weak (34), the kinetics for the inhibition of FVIIa amidolytic activity of hemextin AB complex alone was not studied.

TABLE 2

| [Inhibitor] μM | Vₘₐₓ | Kₘ(mM) | K_{cat}(1/min) |
|---|---|---|---|
| 0.0 | 1.06E−04 | 3.8 | 3518 |
| 0.0125 | 7.37E−05 | 3.9 | 2456 |
| 0.0250 | 5.51E−05 | 3.9 | 1835 |
| 0.05 | 3.75E−05 | 4.0 | 1252 |

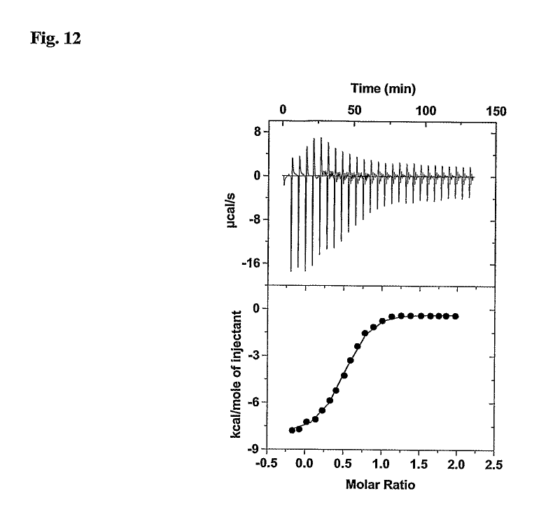

ITC studies—The thermodynamic changes associated with the binding of hemextin AB complex to FVIIa were also monitored (FIG. 12). The binding was exothermic, with ΔH=−7.931 kcal.M⁻¹ for, ΔG =−7.543 kcal.M⁻¹, and ΔS =−1.25 cal.M⁻¹. The calculated K for the binding was 4.11×10⁵M⁻¹

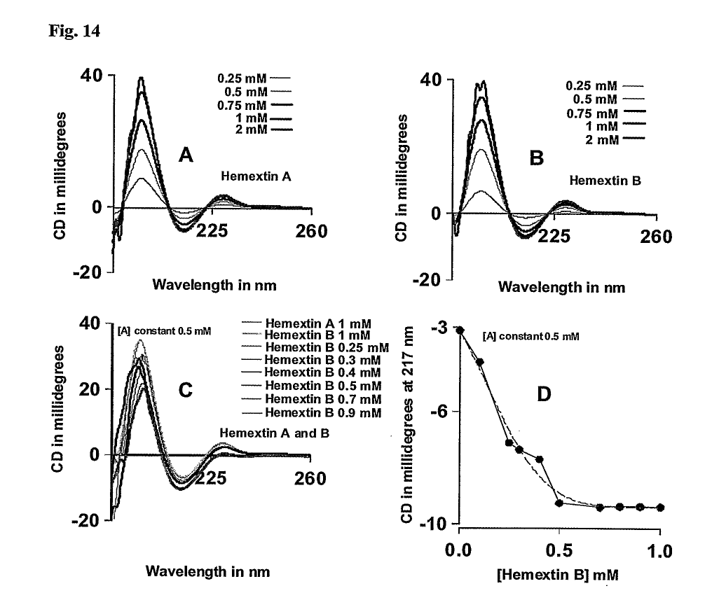

Conformational changes during complex formation—Earlier, it has been shown that hemextin A and hemextin B interact with each other and form a 1:1 tetrameric complex and this complex formation is important for its ability to inhibit FVIIa and clot initiation (74). To study conformational changes associated with hemextin AB complex formation, far UV-CD was used. First, the individual CD spectra of individual hemextins A and B at various concentrations were recorded (FIG. 14, A and B). The CD spectra of hemextin A and hemextin B displayed negative minima at 217 nm and positive maxima at 196 nm, which are due to the π→π*..transition of the amide chromophore and the n→*..transition, respectively, typical of a β-sheet structure (FIG. 14, A and B). However, at higher concentrations, aggregation was observed in both the proteins (FIG. 14, A and B). Next, a titration CD experiment was performed in order to study the complex formation between the two proteins. In this experiment, the concentration of hemextin A was kept constant at 0.5 nM and the conformational changes in hemextin A in the presence of various concentrations of hemextin B was recorded (FIG. 14, C and D). β sheet content increased with the addition of increased amounts of hemextin B. Thus, Hemextin AB complex exhibited a more stable β sheet. No significant change in the spectrum was observed upon further addition of hemextin B after the ratio of concentration of hemextin A to hemextin B reached 1:1 (FIG. 14C). CD studies, therefore, show that hemextin AB complex formation is associated with the stabilization of β sheet conformation and confirmed the 1:1 stoichiometry.

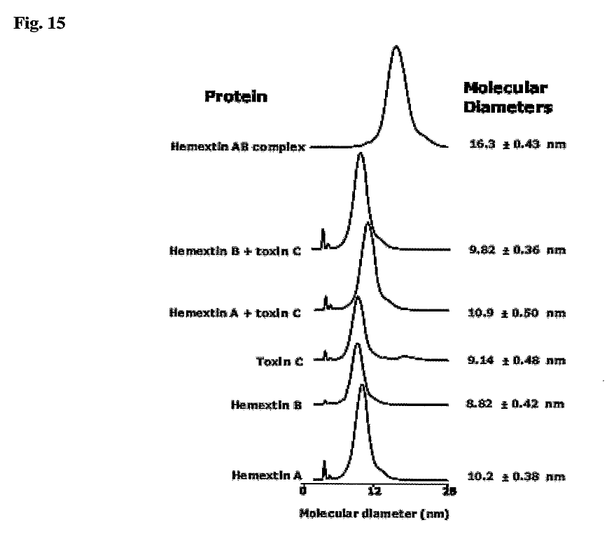

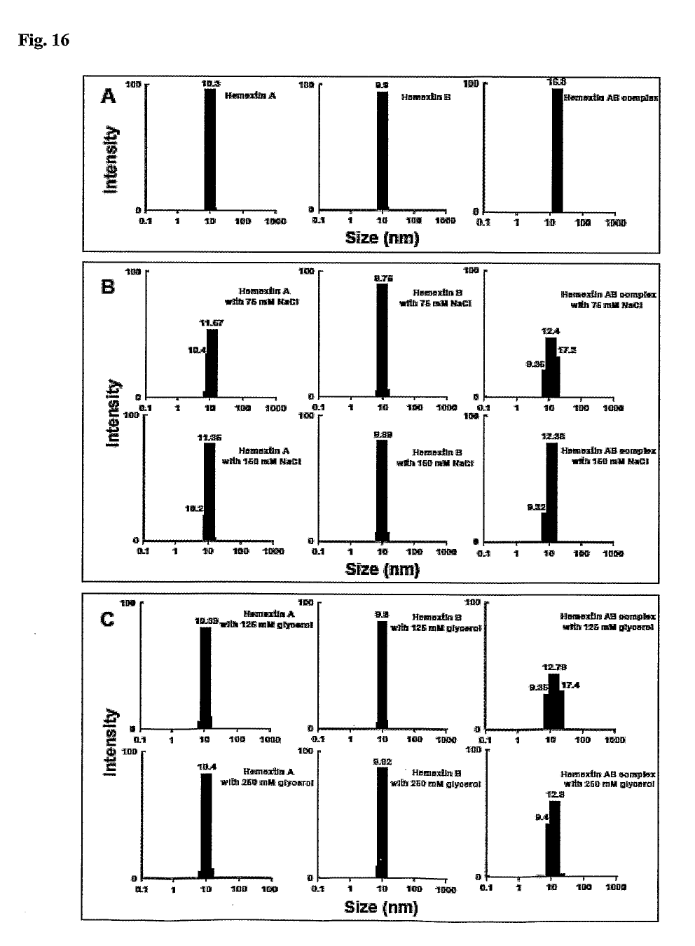

Changes in molecular diameters during complex formation—The diameter of the individual hemextins and hemextin AB complex were determined in both the gas and solution phases. As determined by their electrophoretic mobility in the gas phase using GEMMA, hemextin A and hemextin B show apparent molecular diameters of 10.2±0.38 nm and 8.82±0.42 nm, respectively (FIG. 15). Hemextin AB complex exhibited a larger diameter of 16.3±0.43 nm. Since GEMMA is a considerably new technique employed in studying protein-protein interaction (75), the results were further validated by examining the effect of another protein (toxin C, isolated from the venom of H. haemachatus) on molecular diameters of hemextin A and hemextin B. Toxin C did not affect the anticoagulant activity of hemextin A as determined by prothrombin time assay (data not shown) and did not form a complex with hemextin A. In GEMMA, toxin C at equimolar concentration did not affect the molecular diameter of hemextin A or hemextin B (FIG. 14). The hydrodynamic diameters of the individual hemextins and hemextin AB complex in 50 mM Tris-HCl buffer (pH 7.4) were also determined using DLS. Single scattering populations (unimodal distribution) for hemextin A, hemextin B and hemextin AB complex were observed suggesting the homogeneity of the sample preparations with hydrodynamic diameters of 10.3 nm, 9.9 nm and 16.8 nm, respectively (FIG. 16A). The presence of monodisperse complex (indicated by the narrow size distribution) upon mixing hemextin A and hemextin B suggest the formation of a well-defined complex. The size of the hemextin AB complex is, however, much smaller compared to the estimated size of a tetramer indicating that the complex is a rigid structure (76).

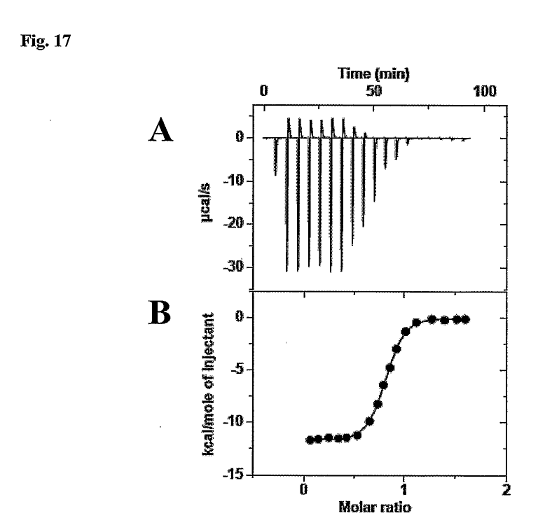

Thermodynamics of hemextin AB complex formation—ITC was used to study the thermodynamics of complex formation. Each injection gave rise to negative (exothermic) heat of reaction (FIG. 17). The binding isotherm fits to a single set of binding sites model, suggesting an equimolar binding between hemextin A and B. The interaction between hemextin A and hemextin B is thermodynamically allowed (as indicated by the negative free energy change) (Table 3). A favorable negative enthalpy but unfavorable negative entropy changes indicate that the complex formation is enthalpically driven. Further, the negative entropy change confirms the formation of a rigid complex, as was indicated by the data obtained by the GEMMA and DLS experiments. The binding constant (Kₐ) of 2.23×10⁶ M⁻¹ was observed for the formation of hemextin AB complex and it falls within Kₐ values for protein-protein interactions in the biologically relevant processes that range from 10⁴ to 10¹⁶ M⁻¹ (70).

TABLE 3

| Temperature | | Kₐ × 10⁶ | ΔH | ΔS | ΔG |
|---|---|---|---|---|---|
| (° C.) | Buffer | (M⁻¹) | (kcal/mole) | (cal/deg.mole) | (kcal/mole) |
| 10 | 50 mM Tris (pH 7.4) | 0.64 | −6.85 | −2.24 | −6.22 |
| 25 | 50 mM Tris (pH 7.4) | 2.07 | −9.92 | −4.43 | −8.6 |
| 37 | 50 mM Tris (pH 7.4) | 2.23 | −11.7 | −8.645 | −9 |
| 45 | 50 mM Tris (pH 7.4) | 1.97 | −13.12 | −12.49 | −9.15 |
| 37 | 50 mM Tris (pH 7.4) + | 0.63 | −10.5 | −7.2 | −8.2 |
| | 35 mM NaCl | | | | |
| 37 | 50 mM Tris (pH 7.4) + | 0.33 | −9.32 | −4.8 | −7.8 |
| | 75 mM NaCl | | | | |
| 37 | 50 mM Tris (pH 7.4) + | 0.02 | −7.31 | −3.82 | −6.12 |
| | 100 mM NaCl | | | | |
| 37 | 50 mM Tris (pH 7.4) + | 0.002 | −5.01 | −1.2 | −4.6 |
| | 150 mM NaCl | | | | |
| 37 | 50 mM Tris (pH 7.4) + | 0.32 | −10.8 | −11.01 | −7.6 |
| | 125 mM glycerol | | | | |
| 37 | 50 mM Tris (pH 7.4) + | 0.2 | −10.5 | −10.6 | −7.2 |
| | 175 mM glycerol | | | | |
| 37 | 50 mM Tris (pH 7.4) + | 0.05 | −9.4 | −10 | −6.4 |
| | 250 mM glycerol | | | | |

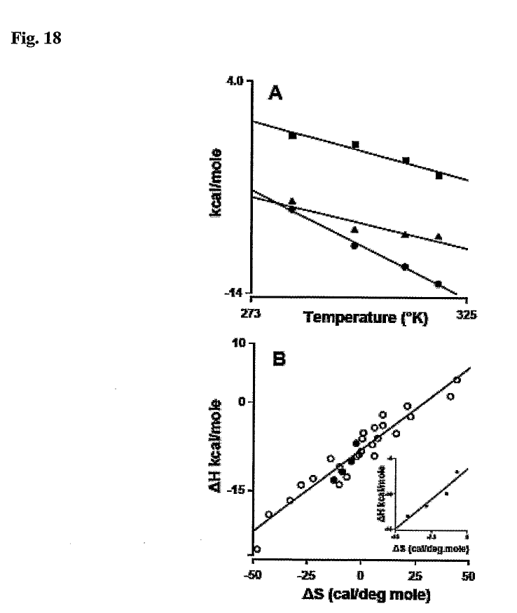

Effect of temperature on complex formation—In several protein-protein and protein-peptide interactions, calorimetric enthalpy could be affected by changes in the experimental temperature. The temperature dependence of the binding of hemextin A to hemextin B was studied over the range of 10-45° C., with the thermodynamic parameters enthalpy (ΔH) entropy (ΔS), and free energy (ΔG) as a function of temperature being shown in FIG. 18A and Table 3. It is clear that the complex formation is enthalpically driven at all temperatures. The temperature dependence data can be used to determine the heat capacity change (ΔCₚ=δΔH/δT) for complex formation. A plot of ΔH versus temperature was linear in this temperature range (FIG. 18A). The slope of the line yields ΔCₚ of −177 cal mol⁻¹ deg⁻¹ for the binding hemextin A and hemextin B. The ΔCₚ for the binding reaction is modest and indicative of a rigid complex formation (77, 78), supporting other experimental observations described above. Also, negative heat capacity changes are typically observed in protein-protein interactions and are attributed to the burial of solvent-accessible hydrophobic surface area (70). A plot of ΔH versus ΔS values for the binding of hemextin A to B at different temperatures shows a slope of ˜1.1 (inset, FIG. 18B), which is common for protein-protein binding processes (79-83), and is due to enthalpy/entropy compensation. This is a direct consequence of a considerably high ΔCₚ value, since (δΔH/δT)ₚ=ΔCₚ and (δ(TΔS)/δT)ₚ=ΔCₚ+ΔS, and then if ΔCₚ>>ΔS, the changes in ΔH and TΔS with temperature will be roughly the same (=ΔCₚ) and will compensate each other. ΔG changes were minimal over the investigated temperature range (FIG. 18A). The values of ΔH and ΔS are always negative, which is again indicative that the binding process of hemextin A to hemextin B is enthalpically favored but entropically unfavored. A linear dependence of ΔH on temperature indicates a two-state binding process with equilibrium between the free and bound forms.

Effect of buffer ionization on complex formation—The observed calorimetric enthalpy is a result of the binding event in addition to all the associated events (water a(di)ssociation, ionization of the components, heats of dilution, heats of mixing, etc). To facilitate binding, residues at the interface may be protonated or deprotonated, resulting in exchange of protons with the buffer. Under such circumstances, as calorimetric enthalpy is dependent on the buffer ionization enthalpy, calorimetric titrations were also performed in phosphate and MOPS buffers at pH 7.4. An increase in the enthalpy change (ΔH_{obs}) (Table 3) was observed with an increase in the buffer ionization enthalpy change (ΔHᵢₒₙ) on the complex formation. A plot of calorimetric enthalpy against ionization enthalpy yielded the number of protons (n_{H}⁺) involved in the interaction, and the binding enthalpy was corrected for protonation effects (ΔH_{bin}) according to the following relationship:

ΔH_{obs}=ΔH_{bin}+n_{H}⁺·ΔHᵢₒₙ (Eq. 4)

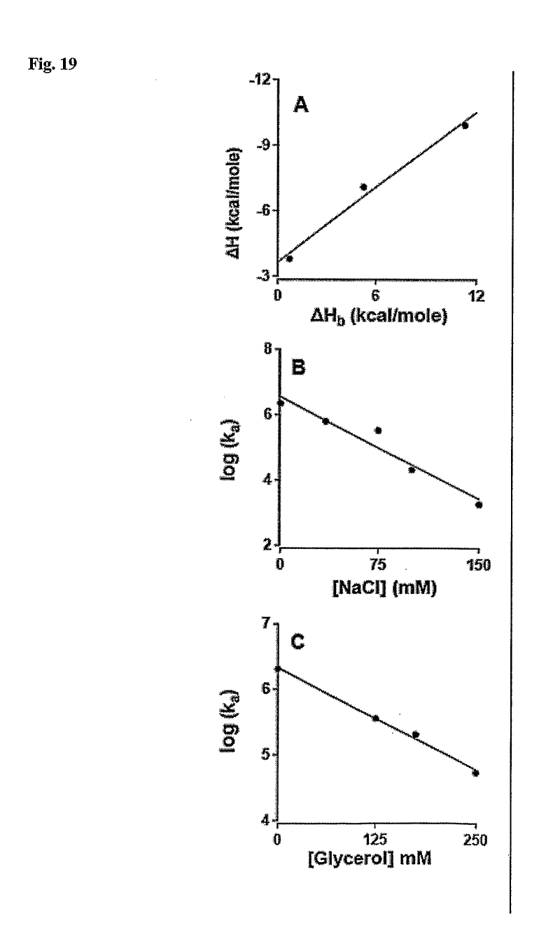

A positive slope indicates propensity for the uptake of protons from the buffer, a negative value indicates propensity for the release of protons into the buffer. The plot (FIG. 19A) yielded an n_{H}⁺ value of −0.57 and a binding enthalpy (ΔH_{bin}) of −3.638 kcal/mole for the complex formation. Thus, the hemextin AB complex formation is associated with a net release of protons into the buffer.

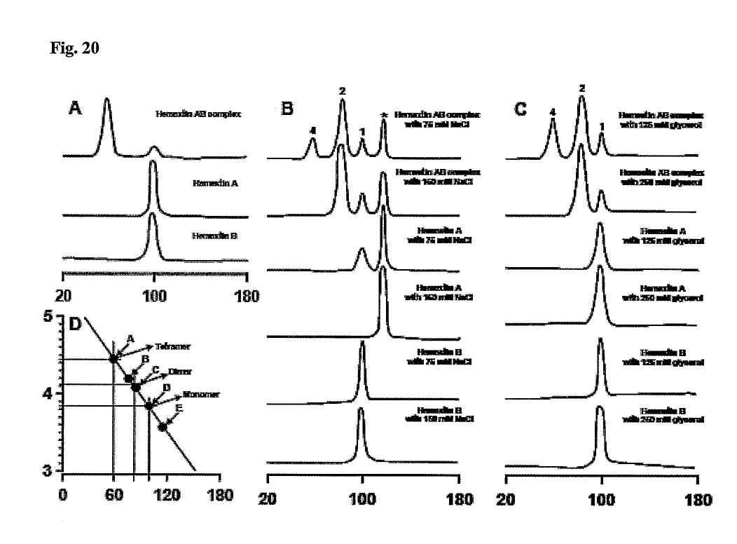

Electrostatic interactions in hemextin AB complex formation—Electrostatic interactions play an important role in protein-protein interactions and provide the specificity to the binding interface. The role of electrostatic interactions in the complex formation was evaluated using ITC, SEC and DLS. Firstly, the binding constant for hemextin AB complex formation was determined by ITC in buffers of increasing ionic strength. The ionic strengths of the buffers were varied by using different concentrations of NaCl. The log Kₐ values for complex formation decreased linearly with increase in NaCl concentration (FIG. 19B, Table 3), showing the probable participation of the electrostatic interactions in complex formation. Secondly, the effect of buffer ionic strength on assembly of the hemextin AB complex was evaluated with the help of SEC. As shown earlier (74), hemextin A and B eluted as a tetrameric complex whereas individual hemextins eluted as monomers (FIG. 20A). To study the role of electrostatic interactions in complex formation, the complex was eluted in buffers containing different concentrations of NaCl. As shown in FIG. 20B, in the presence of 75 mM NaCl the tetramer starts breaking down to dimer. In a buffer of higher ionic strength (NaCl 150 mM) the complex eluted mostly as a dimer and monomer. ESI-MS and HPLC analyses of the dimer peak indicate that it contains both hemextins A and B (data not shown). This observation highlights the probable participation of electrostatic interactions in hemextin AB complex formation. Interestingly, an additional protein peak eluted slower than the monomers indicating hemextin A and/or hemextin B was undergoing a conformational change in buffers with higher ionic strength. Therefore, the elution profiles of individual hemextins A and B in the presence of buffer of high ionic strength were studied. Hemextin A at 75 mM NaCl concentration showed two peaks; a second protein peak eluted slower than the monomer. At 150 mM NaCl concentration hemextin A eluted mostly in the second peak. ESI-MS and HPLC analyses of this second peak show that it is structurally intact hemextin A (data not shown). Thus, the change in the elution profile of hemextin A in the buffer of higher ionic strength hinted a conformational change in the protein, which was confirmed by 1D NMR studies (see below). However, increased ionic strength of the buffer did not have any effect on the elution of hemextin B (FIG. 20, A and B).

The hydrodynamic diameters of the hemextin AB complex and the individual hemextins in buffer solutions of high ionic strength using DLS were also determined (FIG. 16B). At high salt concentrations, the hemextin AB complex exhibits a high polydispersity indicating the presence of a few different species. At 75 mM NaCl concentration, there are at least three different populations; in addition to the monomers and the tetramer, there is an additional population with an apparent molecular diameter of 12.4 nm. Based on the SEC results (FIG. 20B), the 12.4 nm species could be the dimeric hemextin AB complex. As expected, the population of 12.4 nm species increases when the concentration of NaCl is increased to 150 mM (FIG. 16B). Thus, DLS data also suggests the dissociation of the tetrameric complex to a dimer. Interestingly, polydispersity was also observed in case of hemextin A in buffers of high ionic strength (FIG. 16B). There is an additional population of 11.57 nm sized particle, in addition to its native size of 10.4 nm. Based on the SEC (FIG. 20B) and 1D-NMR (see below), the 11.57 nm species may represent the conformationally altered form of hemextin A. No change in the hydrodynamic diameter of hemextin B was observed with the change in buffer ionic strength (FIG. 16B). These studies show that the tetrameric complex breaks down to dimer and monomer with the increasing concentration. This breakdown could be due to the interference in electrostatic interactions between the subunits and/or the change in conformation of hemextin A.

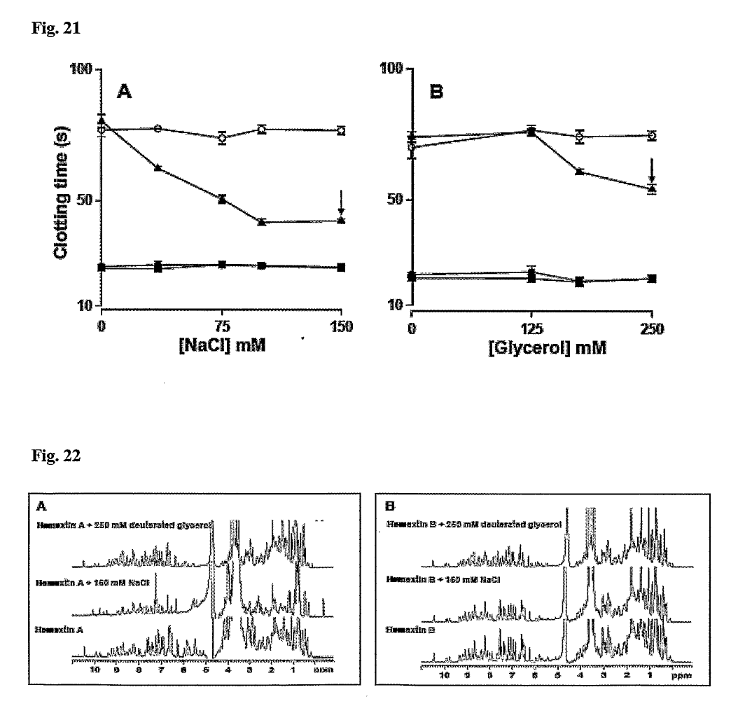

To understand the implication of the change in conformation of hemextin A and the breakdown of tetrameric complex, the anticoagulant activity of the complex and the individual hemextins in buffers of high ionic strengths was monitored. Anticoagulant activity of the hemextin AB complex decreased with the increase in ionic strength up to 100 mM NaCl (FIG. 21A). However, further increase in the salt concentration did not significantly affect the anticoagulant activity. At 150 mM NaCl concentration the complex exists as a mixture of a dimer, monomer(s) and conformationally altered hemextin A (FIG. 20B). However, the higher concentration of NaCl did not affect the anticoagulant activity of hemextin A (FIG. 21A). Thus despite the change in the conformation (see below), hemextin A retains its anticoagulant activity. Therefore, the remaining anticoagulant activity observed at 150 mM NaCl concentration is due to the presence of hemextin A. From these results, it may be concluded that the dimer formed at high salt concentrations does not have any significant anticoagulant activity.

Hydrophobic interactions in hemextin AB complex formation—Hydrophobic interactions act as the driving forces in the complex formation. The importance of hydrophobic interactions in the complex formation using ITC, SEC and DLS was also evaluated. ITC experiments were performed in buffers containing increasing concentrations of glycerol. Glycerol forms a ‘hydration’ layer around the protein, thereby inhibiting hydrophobic interactions. A decrease in the association constant was observed with the increase in glycerol concentration (FIG. 19C and Table 3), showing the importance of hydrophobic interactions in the complex formation. The elution of hemextin AB complex in buffers containing glycerol on a Superdex 75 column was monitored (FIG. 20C). In buffers containing high glycerol concentration, the tetramer breaks down to dimer and monomers. ESI-MS and HPLC analyses of the dimer peak indicate that it contains both hemextins A and B (data not shown). However, no additional peak corresponding to the altered conformation of hemextin A was observed. The elution of individual hemextins remained unaltered in the presence of glycerol (FIG. 20C). The breakdown of hemextin AB complex in the presence of glycerol was also observed in the DLS studies (FIG. 16C). At 125 mM glycerol concentration, an additional population of 12.8 nm sized species was observed in addition to the monomers and the tetrameric complex. Based on SEC studies, it is proposed that the 12.8 nm species is a dimer. The 12.8 nm species increases with the increase in glycerol concentration (FIG. 16C). It is important to note that the apparent molecular diameter of this dimer is different from the dimer formed in the presence of high ionic buffers (12.8 nm versus 12.4 nm; FIGS. 16B and 16C). (As GEMMA works on the principle of nano-ESI, the molecular diameters in buffers containing high salt and glycerol were not determined using this technique.) No polydispersity was observed in the case of individual hemextins in the presence of glycerol (FIG. 16C). These studies show that hydrophobic interactions play an important in the formation of hemextin AB complex.

To understand the implication of the breakdown of hemextin AB complex, its anticoagulant activity and that of the individual hemextins in buffers containing different concentrations of glycerol were monitored. The anticoagulant activity of hemextin AB complex decreased with the increase in glycerol concentration (FIG. 21B). At 125 mM glycerol concentration there is no decrease in the anticoagulant activity of the complex. At 250 mM glycerol concentration though there is a decrease in the anticoagulant activity, but it is higher than that of anticoagulant effect of hemextin A alone. Further, glycerol did not affect the anticoagulant activity of individual hemextins (FIG. 21B). SEC studies show that at 250 mM glycerol concentration the complex mostly exists as a mixture of dimer and monomers (FIG. 20C). As the anticoagulant activity is higher than that of hemextin A alone, the dimer observed at 250 mM glycerol concentration exhibits anticoagulant activity higher than hemextin A alone but lower than the tetramer. Thus, the dimer formed in the presence of glycerol is different from the dimer formed in the presence of salt; the former dimer showed an increased anticoagulant activity compared to hemextin A alone, whereas the latter dimer did not.

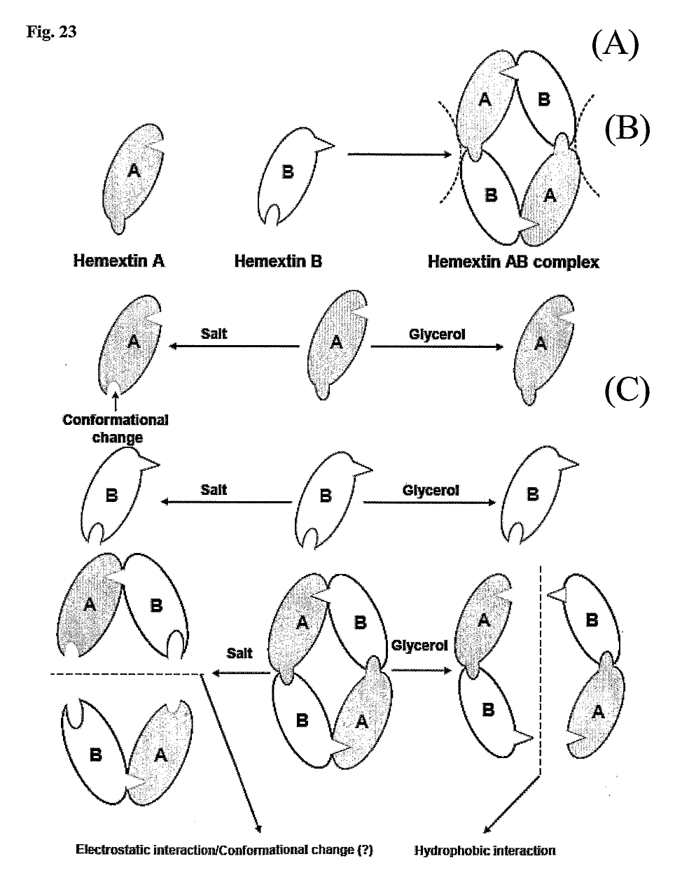

Effect of buffer conditions on the conformation of hemextins—Earlier studies using SEC (FIG. 20B) and DLS (FIG. 16B) indicated that hemextin A undergoes a conformational change in the presence of salt. Therefore, 1D-NMR studies were conducted to study the conformation of hemextins A and B under different buffer conditions (FIG. 22). In the presence of NaCl, there is a decrease in the number of Hα resonance peaks between 4.8 ppm and 6 ppm (FIG. 22A). These chemical shifts contribute to the inter-residue NOE cross peaks between Hα of different amino acid residues forming anti-parallel β sheet structure typically observed in all three-finger toxins (84). Thus, a decrease in the β sheet content of hemextin A is observed in the presence of NaCl. In addition, there are several changes in the chemical shifts of side chains. A notable change is a highly shielded methyl peak which appears at the negative chemical shift value (−0.38 ppm) in the presence of salt. These observations strongly support conformational changes in hemextin A in the presence of NaCl. The overall dispersion of 1D proton NMR spectra of hemextin A in the presence of glycerol (deuterated) remains the same with the subtle changes in the amide region (FIG. 22A). Thus, hemextin A did not undergo any significant conformational change upon the addition of glycerol. Similar studies with hemextin B show that it did not undergo any significant conformational change in the presence of NaCl or glycerol (FIG. 22B) since there is almost one to one match for the spectral frequencies.

### DISCUSSION

Initiation of blood coagulation during injury or trauma is essential for the survival of the organism. However, the formation of unwanted clots has detrimental or debilitating effects and hence the need for anticoagulant therapies. Current anticoagulants used for treating these disorders are non-specific and have a narrow therapeutic range necessitating careful laboratory monitoring to achieve optimal efficacy and minimize bleeding. This is further complicated by other factors such as dietary intake (35). Therefore, novel anticoagulant and antiplatelet agents are being sought after. Since the FVIIa is the key initiator of blood coagulation and is present in the plasma milieu at very low concentrations, it beckons to be an attractive drug target for the design and development of anticoagulants.

So far, only two proteins that are known to specifically inhibit the TF-FVIIa complex have been well characterized, namely, tissue factor pathway inhibitor (TFPI) and nematode anticoagulant peptide c2 (NAPc2). TFPI is an endogenous inhibitor of this complex (36), whereas NAPc2 is an exogenous inhibitor isolated from canine hookworm, Ancylostoma caninum (37). TFPI is a 42 kDa plasma glycoprotein consisting of three tandem Kunitz type domains. The first and the second units inhibit TF-FVIIa and FXa respectively. The third Kunitz domain and the C-terminal basic region of the molecule have heparin binding sites (38). The anticoagulant action of TFPI is a two-stage process. The second Kunitz domain binds first to a molecule of FXa and deactivates it. The first domain then rapidly binds to an adjacent TF-FVIIa complex, preventing further activation of FX (39-41). On the other hand, NAPc2 is an 8 kDa short polypeptide. Its mechanism of action requires prerequisite binding to FXa or zymogen FX to form a binary complex prior to its interaction and inhibition of membrane-bound TF-FVIIa (42). Therefore, despite the structural differences, both the inhibitors form a quaternary complex with TF-FVIIa-FXa. However, in both complexes, the active site of FVIIa is occupied by the respective inhibitors and is not accessible.

Due to lack of natural inhibitors that specifically interfere in the FVIIa activity, a number of artificial inhibitors have been designed and developed. They include proteins that block the association of TF and FVIIa, such as antibodies against TF or FVIIa, TFAA (a mutant TF with reduced cofactor function for FX), FFR-VIIa (inactivated form of FVIIa with fivefold higher affinity for TF than that of native FVIIa) and peptides derived from TF or FVIIa (43-50). In addition, two series of peptide exosite inhibitors were selected from phage-display libraries for their ability to bind to TF-FVIIa complex (43, 44). They bind to two distinct exosites on the serine protease domain of FVIIa, and exhibit steric and allosteric inhibition (46). Although both peptide classes were potent and selective inhibitors of TF-FVIIa complex, they fail to inhibit 100% activity even at saturating concentrations. This was overcome by either the fusion of the two peptides (47), or using a protease switch with substrate phage (45). A number of synthetic compounds have also been designed as the active site inhibitor of FVIIa as well as TF-FVIIa complex (48, 51-54). Recently, a number of napthylamidines have been reported to have FVIIa inhibitory activity. They were synthesized by the coupling of amidinobenzaldehyde analogs to a polystyrene resin. However, apart from inhibiting FVIIa activity, these synthetic compounds nonspecifically inhibited the activity of other blood coagulation serine proteases (55).

The isolation and characterization of two proteins—hemextin A and hemextin B from the venom of H. haemachatus that synergistically induce potent anticoagulant activity are reported herein. Both hemextins A and B belong to the three-finger family of snake venom proteins (FIG. 3). Individually, only hemextin A exhibited mild anticoagulant activity, whereas hemextin B has no anticoagulant activity (FIG. 4A). However, hemextin B synergistically enhances the anticoagulant activity of hemextin A and their complex exhibits potent anticoagulant activity. The increase in the anticoagulant potency of hemextin A in the presence of hemextin B (FIG. 4A) indicated probable complex formation between the two proteins. It was shown that the 1:1 complex formation is important for potent anticoagulant activity using prothrombin time assay (FIG. 4B). The complex formation was further confirmed by gel filtration chromatography (FIG. 5).

Using the “dissection approach” (32, 33), the site of anticoagulant action of hemextin A and its synergistic complex were identified (FIG. 6A). Using three common clotting time assays, hemextin A and hemextin AB complex were shown to inhibit the extrinsic tenase complex but not other steps in the extrinsic pathway (FIG. 6B-D). These results were further confirmed by studying the effect of hemextin A and its complex on the reconstituted TF-FVIIa complex. Both hemextin AB complex and hemextin A inhibit the FXa formation by the reconstituted extrinsic tenase complex (FIG. 7A). Interestingly, hemextin A and hemextin AB complex inhibit the amidolytic activity of FVIIa both in the presence and in the absence of sTF with an IC₅₀ of ˜100 nM and ˜105 nM respectively (FIGS. 8A and B). Similar IC₅₀ values may be indicative of the fact that hemextin A and hemextin AB complex do not bind to the cofactor binding site of FVIIa. The inhibitory activity of hemextin A and hemextin AB complex may not be due to nonspecific interaction of hemextin A or its complex with the phospholipids in the extrinsic tenase complex, as indicated by their inability to prolong the Stypven time, since they failed to inhibit the prothrombinase complex, which is also formed on the phospholipid surfaces. This was further confirmed by determining the inhibitory activity of hemextin A and hemextin AB complex on the amidolytic of reconstituted extrinsic tenase complex using sTF and FVIIa (FIG. 8A). Further, hemextin A and hemextin AB complex inhibited amidolytic activity of FVIIa. Hemextin B, however, did not exhibit any inhibitory activity in the absence of hemextin A. To further characterize the inhibitory properties and determine the specificity of inhibition, hemextins A and B and hemextin AB complex were screened against 12 serine proteases. In addition to FVIIa and its complexes, hemextin A and hemextin AB complex inhibited the amidolytic activity of only kallikrein in a dose-dependant manner. However, the IC₅₀ for the inhibition of kallikrein was ˜5 μM, in contrast to that of FVIIa/FVIIa-TF/FVIIa-sTF which was ˜100 nM. Kinetic studies revealed that hemextin AB complex is a non-competitive inhibitor of FVIIa-sTF complex with a Kᵢ of 25 nM. Using ITC studies, it was shown that hemextin AB complex directly interacts with FVIIa. The binding interaction between FVIIa and hemextin AB complex is associated with a negative change in free energy indicating that this complex formation is favored. Negative change in entropy observed with the binding indicates the formation of a tightly folded complex between the two moieties (56). Thus, these data strongly indicate that hemextin AB complex is a highly specific natural inhibitor of FVIIa.

Some other anticoagulants from snake venoms also inhibit extrinsic tenase complex. However, they are not as specific. For example, CM IV, a strongly anticoagulant phospholipase A₂ (PLA₂) from Naja nigricollis venom prolongs coagulation by inhibiting two successive steps in the coagulation cascade. It inhibits the TF-FVIIa complex by both enzymatic and nonenzymatic mechanisms (57), whereas it inhibits the prothrombinase complex only by the nonenzymatic mechanism (58, 59). Hemextin A and its synergistic complex are the first reported specific inhibitors of FVIIa isolated from snake venom.

Similar dose-dependent inhibition of TF-FVIIa complex and FVIIa indicates that hemextin AB complex neither requires TF for its inhibitory activity nor interferes in the binding of TF to FVIIa. Unlike TFPI and NAPC2, it also does not use FXa as a scaffold to bind to FVIIa and thus does not require FX or FXa to inhibit FVIIa. Further, TFPI and NAPC2 bind to the active site of FVIIa. In contrast, hemextin AB complex is a noncompetitive inhibitor and hence the does not interact with FVIIa through its active site. Thus, hemextin A and hemextin AB complex are novel inhibitors of FVIIa and TF-FVII complex.

CD studies showed that the complex formation leads to the stabilization of β-sheeted structure (FIG. 14). The interaction also results in the formation of a rigid structure. This is reflected in the conformational entropy penalty associated with the formation of the hemextin AB complex (Table 3). GEMMA and DLS studies show that in both gas and liquid phases, there is an increase in the apparent molecular diameters during the complex formation (FIGS. 15 and 16). The molecular diameters from these techniques are nearly identical. However, the apparent molecular dimensions are fairly larger than the theoretical diameter estimated for a native protein and much smaller than the estimated length of the proteins in completely “extended conformation” (85, 86). Such an anomaly could be due to the non-globular conformation of the proteins (87).

ITC permits the study of macromolecular interactions in solution and is the only technique that can resolve the enthalpic and entropic components of binding affinity and hence the difference in the Gibbs free energy between the initial and final states (88-90). The interaction between hemextin A and hemextin B is characterized by favorable negative changes in ΔH. Thus, Van der Waals interactions and hydrogen bonds may play an important role in the complex formation.

The energetic parameters obtained for the interaction between hemextin A and hemextin B showed a strong dependence on the experimental temperature. Despite the differences observed in the enthalpy and entropy change with temperature, the free energy changes remained minimal (FIG. 18A), suggesting enthalpy-entropy compensation. This phenomenon is a universal feature for protein-peptide interactions, where weak molecular interactions undergo constant rearrangements to realize a lower free energy of binding (91-93). FIG. 18B shows the correlation between entropy and enthalpy (r²=0.956) for a range of interacting protein-protein systems. The data for hemextin AB complex formation falls well along this correlation line.

According to the laws of thermodynamics, the temperature dependence of ΔH and ΔS results from changes in ΔCp. In almost all association processes with proteins, ΔCp has a negative sign if the free components are the reference state (94). In hemextin AB complex formation a ΔCp of the binding −177 cal mol⁻¹ deg⁻¹ was observed.

Changes in the negative ΔCp indicate a reduction in the nonpolar solvent-accessible surface area, as explained by the following equation (95),

ΔCₚ=0.45(ΔASAₙₒₙₚₒₗ)−0.26(ΔASAₚₒₗ)cal/molK (Eq. 5)

where ΔASAₚₒₗ and ΔASAₙₒₙₚₒₗ are the change in the polar- and non-polar-accessible surface areas respectively. Large negative ΔCp changes have been observed in protein-peptide interactions, in protein folding governed by hydrophobic effect (96, 97), and in complex formation associated with the burial of solvent-exposed hydrophobic residues (80, 81, 98, 99). In contrast, burial of polar surface area contributes to a weakly positive ΔCₚ. The ΔCp change for hemextin AB complex formation is negative, albeit weaker than that are typically observed in protein-protein interactions (70). Negative ΔCp supports the classical model of hydrophobic effect proposed by Tanford (100) and is accompanied by a reorganization of the solvent molecules, thus increasing solvation entropy. This process contradicts the unfavorable ΔS observed during hemextin A-hemextin B interaction. However, this phenomenon is not uncommon in protein-protein interactions (101-110). The observed unfavorable ΔS could be due to possible conformational changes occurring in hemextin A and/or hemextin B upon binding (FIG. 14) and/or due to the binding of water molecules at the interface of the interacting proteins. Ladbury et al. have suggested that the restriction of degrees of freedom of water molecules within highly hydrated specific interfaces could also make a substantial negative contribution to the ΔCₚ (111), as observed in several protein-protein complexes (80, 112-114). Water molecules at the interface can act as molecular bridges mediating interactions between proteins and ligands through hydrogen bonds (113, 115) or change the shape complementarities between the protein and ligand surfaces (116, 117).

The hemextin AB tetramer breaks down in to a dimer and monomers in the presence of high salt (FIGS. 19B, 20B, 16B, 21A and Table 3). Thus, one would intuitively suspect the participation of the electrostatic interactions in the complex formation. However, when binding is dominated by interactions between polar groups there will be a weak positive ΔCₚ. In contrast, the observed negative value for ΔCₚ is consistent with the formation of a binding interface containing “bridging” hydrogen bonds formed by sequestered water molecules or with the conformational changes occurring upon binding. As hemextin A undergoes conformational changes in the presence of salt (FIGS. 22A and 23A), the dissociation of the tetramer in a buffer of high ionic strength is possibly due to the conformational change in hemextin A. However, the role of electrostatic interactions in complex formation cannot be ruled out.

The hemextin AB tetramer also breaks down in to a dimer and monomers in the presence of glycerol (FIGS. 20C, 19C, 16C and 21B). Thus hydrophobic interactions play an important role in the complex formation. This is also supported by the observed negative ΔCₚ changes in the ITC experiments at different temperatures (FIG. 18A). Further, the breakdown is not due to the conformational changes in hemextins as glycerol does not affect the conformations of the individual hemextins (FIGS. 22 and 23). Therefore, hydrophobic interactions may provide the driving force for the complex formation.

Model for formation of hemextin AB complex—Two molecules each of hemextin A and B form a tetrameric complex in Tris-HCl buffer. The formation of this synergistic complex is important for its anticoagulant activity. As described earlier, hemextin AB dimer in high salt is different from the dimer formed in the presence of glycerol. The former dimer has an apparent molecular diameter of 12.4 nm and lacks anticoagulant activity, whereas the latter dimer has an apparent molecular diameter of 12.8 nm and exhibits slightly higher anticoagulant effects (FIG. 23). Thus, the breakdown of tetramer to dimer probably occurs in two different planes of interaction between hemextin A and B. One plane is sensitive to the ionic strength of its surroundings while the other is sensitive to glycerol (FIG. 23). Further, in the presence of salt, hemextin A undergoes a conformational change (FIG. 23) which may interfere in the tetramer formation. The dimer formed under high ionic conditions lacks the anticoagulant site (marked by a dotted semicircle in FIG. 23). In contrast, hydrophobic interactions are predominant in the second plane. Therefore, glycerol dissociates the tetramer into dimers. However, in this case only minor changes occur in the anticoagulant site of the complex (as shown in FIG. 23) and hence the resultant dimer is active. The tetramer formation most likely stabilizes the anticoagulant site of hemextin A.

The complex formation and synergism among snake venom proteins is well known, particularly among presynaptic neurotoxins. Several snake venom complexes are—crotoxin from Crotalus durissus terrificus (60), taipoxin from Oxyuranus scutellatus (61), rhodocetin from Calloselasma rhodostoma (64), group C prothrombin activators from Australian snakes (65-67). Crotoxin isolated from Crotalus durissus terrificus venom contains two subunits; the basic subunit is a PLA₂ enzyme whereas the acidic subunit is catalytically inactive (although it is derived from a PLA₂-like protein) (60). Individually only the basic subunit is slightly toxic, while the complex exhibits potent toxicity. The acidic subunit appears to act like a chaperone and enhances the specific binding of the basic subunit to the presynaptic site. Similarly, other presynaptic neurotoxins, such as taipoxin from Oxyuranus scutellatus (61) and textilotoxin from Pseudonaja textilis (62) venoms contain three and four subunits, respectively. All the subunits are structurally similar to PLA₂ enzymes. The noncovalent interactions between the subunits of these toxins are important for their potent toxicity. Thus, a number of snake venom presynaptic toxins are protein complexes with PLA₂ as an integral part. Taicatoxin another protein complex isolated from O. scutellatus venom blocks calcium channels, and it has PLA₂, proteinase inhibitor and neurotoxin (a three-finger toxin) subunits (63). There are only a few non-covalent protein complexes in snake venoms that do not contain PLA₂ as an integral part. For example, rhodocetin, an antiplatelet protein complex from Calloselasma rhodostoma venom, contains two subunits showing structural similarity to C-type lectins (64). Group C prothrombin activators from Australian snakes are procoagulant protein complexes, which are structurally and functionally similar to mammalian blood coagulation FXa-FVa complex (65-67). Rhodocetin is an antiplatelet protein complex which is a heterodimer of C-type lectin related proteins (61). Pseutarin C is a procoagulant complex which is structurally and functionally similar to mammalian FXa-FVa complex (65-66). In the remaining cases, the respective subunits are held together by yet-to-be-characterized non-covalent interactions. Hemextin AB complex is the first anticoagulant complex isolated from snake venoms in which the anticoagulant activity of hemextin A is potentiated by its synergistic interaction with hemextin B (74). It specifically and non-competitively inhibits FVIIa, without the requirement of FX scaffold. Thus, this is the first known natural proteinaceous inhibitor of FVIIa. Structurally it is the only known tetrameric complex formed by two three-finger toxins (74). As the complex formation is essential for the synergistic inhibition of the clot initiation, elucidation of the molecular interactions that govern the formation of this unique complex is important.

In summary, a unique anticoagulant protein complex from snake venom that specifically and non-competitively inhibits the activity of FVIIa activity is described herein. The results strongly support that the interaction between hemextin A and B is essential for potent anticoagulant activity. The unique protein-protein complex between two closely related three-finger toxins were characterized using various biophysical techniques. Circular dichroism studies showed that the complex formation leads to the stabilization of β sheet. Hemextin AB complex is rigid and its formation is enthalpically driven. The negative value for heat capacity indicates the presence of hydrogen bonds and the occurrence of conformational change(s). Hydrophobic interactions primarily drive the process of complex formation, though the stability of the complex is also dependant on the ionic strength of its surroundings. The tetramer dissociates into a dimer in the presence of salt as well as glycerol. The dimer formed in the presence of salt appears to be different from that formed in the presence of glycerol; their apparent molecular diameters are different and they exhibit different anticoagulant properties. The dissociation of the complex in the presence of salt is probably due to the conformational change in hemextin A. Based on the results, a model to define the assembly of hemextin AB complex was proposed.

Advantageously, this new anticoagulant may facilitate development of different strategies and therapeutic agents to inhibit the initiation step in blood coagulation. This study will also enable better understanding of the structure-function relationships of this protein complex.

It will be understood that the invention has been described by way of example only and modifications may be made whilst remaining within the scope and spirit of the invention.

### REFERENCES

1. Davie, E. W., Fujikawa, K., and Kisiel, W. (1991) Biochemistry 30, 10363-10370
2. Mann, K. G., Butenas, S., and Brummel, K. (2003) Arterioscler. Thromb. Vasc. Biol. 23, 17-25
3. Davie, E. W. (1995) Thromb. Haemost. 74, 1-6
4. Rapaport, S. I. and Rao, L. V. (1995) Thromb. Haemost. 74, 7-17
5. Morrissey, J. H., Macik, B. G., Neuenschwander, P. F., and Comp, P. C. (1993) Blood 81, 734-744
6. Nemerson, Y. (1988) Blood 71, 1-8
7. Mann, K. G., Butenas, S., and Brummel, K. (2003) Arterioscler. Thromb. Vasc. Biol. 23, 17-25
8. Gustafsson, D., Bylund, R., Antonsson, T., Nilsson, I., Nystrom, J. E., Eriksson, U., Bredberg, U., and Teger-Nilsson, A. C. (2004) Nat. Rev. Drug. Discov. 3, 649-659
9. Hirsh, J. (1991) N. Engl. J. Med. 324, 1565-1574
10. Hirsh, J. (1991) N. Engl. J. Med. 324, 1865-1875
11. Hirsh, J. and Weitz, J. I. (1999) Lancet 353, 1431-1436
12. Moll, S, and Roberts, H. R. (2002) Semin. Hematol. 39, 145-157
13. Hirsh, J. (2001) Am. Heart J. 142, S3-S8
14. Morrissey, J. H. (2001) Thromb. Haemost. 86, 66-74
15. Markland, F. S. (1998) Toxicon 36, 1749-1800
16. Higuchi, S., Murayama, N., Saguchi, K., Ohi, H., Fujita, Y., Camargo, A. C., Ogawa, T., Deshimaru, M., and Ohno, M. (1999) Immunopharmacology 44, 129-135
17. O'Shea, J. C. and Tcheng, J. E. (2002) Expert. Opin. Pharmacother. 3, 1199-1210
18. Marcinkiewicz, C. (2005) Curr. Pharm. Des 11, 815-827
19. Huang, F. and Hong, E. (2004) Curr. Med. Chem. Cardiovasc. Hematol. Agents 2, 187-196
20. Plosker, G. L. and Ibbotson, T. (2003) Pharmacoeconomics. 21, 885-912
21. Kondo, K. and Umemura, K. (2002) Clin. Pharmacokinet. 41, 187-195
22. McClellan, K. J. and Goa, K. L. (1998) Drugs 56, 1067-1080
23. Sherman, D. G. (2002) Curr. Med. Res. Opin. 18 Suppl 2, s48-s52
24. Joseph, J. S., Chung, M. C., Jeyaseelan, K., and Kini, R. M. (1999) Blood 94, 621-631
25. Langdell R D, Wagner R H, and Brinkhous K M (1953) J. Lab. Clin. Med. 41, 637-647
26. Quick A J. (1935) J. Biol. Chem. 109, 73-74
27. Hougie (1956) Proc. Soc. Exp. Biol. Med. 98, 570-573
28. Jim, R. (1957) J. Lab. Clin. Med. 50, 45-60
29. Tomokiyo, K., Yano, H., Imamura, M., Nakano, Y., Nakagaki, T., Ogata, Y., Terano, T., Miyamoto, S., and Funatsu, A. (2003) Vox Sang 84, 54-64
30. Shigematsu, Y., Miyata, T., Higashi, S., Miki, T., Sadler, J. E., and Iwanaga, S. (1992) J. Biol. Chem. 267, 21329-21337
31. Condrea, E., Fletcher, J. E., Rapuano, B. E., Yang, C. C., and Rosenberg, P. (1981) Toxicon 19, 61-71
32. Kini, R. M. and Banerjee, Y. (2005) J. Thromb. Haemost. 3, 170-171
33. Stefansson, S., Kini, R. M., and Evans, H. J. (1989) Thromb. Res. 55, 481-491
34. Maun, H. R., Eigenbrot, C., Raab, H., Arnott, D., Phu, L., Bullens, S., and Lazarus, R. A. (2005) Protein Sci. 14, 1171-1180
35. Goolsby, M. J. (2002) J. Am. Acad. Nurse Pract. 14, 16-18
36. Lindhout, T., Franssen, J., and Willems, G. (1995) Thromb. Haemost. 74, 910-915
37. Lee, A. Y. and Vlasuk, G. P. (2003) J. Intern. Med. 254, 313-321
38. Kato, H. (2002) Arterioscler. Thromb. Vasc. Biol. 22, 539-548
39. Baugh, R. J., Broze, G. J., Jr., and Krishnaswamy, S. (1998) J. Biol. Chem. 273, 4378-4386
40. Broze, G. J., Jr. and Miletich, J. P. (1987) Blood 69, 150-155
41. Sanders, N. L., Bajaj, S. P., Zivelin, A., and Rapaport, S. I. (1985) Blood 66, 204-212
42. Lee, A. Y. and Vlasuk, G. P. (2003) J. Intern. Med. 254, 313-321
43. Dennis, M. S., Eigenbrot, C., Skelton, N. J., Ultsch, M. H., Santell, L., Dwyer, M. A., O'Connell, M. P., and Lazarus, R. A. (2000) Nature 404, 465-470
44. Dennis, M. S., Roberge, M., Quan, C., and Lazarus, R. A. (2001) Biochemistry 40, 9513-9521
45. Maun, H. R., Eigenbrot, C., and Lazarus, R. A. (2003) J. Biol. Chem. 278, 21823-21830
46. Roberge, M., Santell, L., Dennis, M. S., Eigenbrot, C., Dwyer, M. A., and Lazarus, R. A. (2001) Biochemistry 40, 9522-9531
47. Roberge, M., Peek, M., Kirchhofer, D., Dennis, M. S., and Lazarus, R. A. (2002) Biochem. J. 363, 387-393
48. Sorensen, B. B., Persson, E., Freskgard, P. O., Kjalke, M., Ezban, M., Williams, T., and Rao, L. V. (1997) J. Biol. Chem. 272, 11863-11868
49. Hirsh, J., O'Donnell, M., and Weitz, J. I. (2005) Blood 105, 453-463
50. Johnson, K. and Hung, D. (1998) Coron. Artery Dis. 9, 83-87
51. Uchiba, M., Okajima, K., Abe, H., Okabe, H., and Takatsuki, K. (1994) Thromb. Res. 74,
52. Uchiba, M., Okajima, K., Murakami, K., Okabe, H., and Takatsuki, K. (1995) Thromb. Res.
53. Lazarus, R. A., Olivero, A. G., Eigenbrot, C., and Kirchhofer, D. (2004) Curr. Med. Chem. 11, 2275-2290
54. Olivero, A. G., Eigenbrot, C., Goldsmith, R., Robarge, K., Artis, D. R., Flygare, J., Rawson, T., Sutherlin, D. P., Kadkhodayan, S., Beresini, M., Elliott, L. O., Deguzman, G. G., Banner, D. W., Ultsch, M., Marzec, U., Hanson, S. R., Refino, C., Bunting, S., and Kirchhofer, D. (2005) J. Biol. Chem. 280, 9160-9169
55. Buckman, B. O., Chou, Y. L., McCarrick, M., Liang, A., Lentz, D., Mohan, R., Morrissey, M. M., Shaw, K. J., Trinh, L., and Light, D. R. (2005) Bioorg. Med. Chem. Lett. 15, 2249-2252
56. Barry, J. (1976) Curr. Top. Mol. Endocrinol. 3, 451-473
57. Kini, R. M. and Evans, H. J. (1995) Toxicon 33, 1585-1590
58. Stefansson, S., Kini, R. M., and Evans, H. J. (1990) Biochemistry 29, 7742-7746
59. Kerns, R. T., Kini, R. M., Stefansson, S., and Evans, H. J. (1999) Arch. Biochem. Biophys. 369, 107-113
60. Habermann, E. and Breithaupt, H. (1978) Toxicon 16, 19-30
61. Doorty, K. B., Bevan, S., Wadsworth, J. D., and Strong, P. N. (1997) J. Biol. Chem. 272, 19925-19930
62. Su, M. J., Coulter, A. R., Sutherland, S. K., and Chang, C. C. (1983) Toxicon 21, 143-151
63. Possani, L. D., Martin, B. M., Yatani, A., Mochca-Morales, J., Zamudio, F. Z., Gurrola, G. B., and Brown, A. M. (1992) Toxicon 30, 1343-1364
64. Wang, R., Kini, R. M., and Chung, M. C. (1999) Biochemistry 38, 7584-7593
65. Rao, V. S, and Kini, R. M. (2002) Thromb. Haemost. 88, 611-619
66. Rao, V. S., Swarup, S., and Kini, R. M. (2003) Blood 102, 1347-1354
67. Rao, V. S., Swarup, S., and Kini, R. M. (2004) Thromb. Haemost. 92, 509-521
68. Ye, H. and Wu, H. (2000) Proc. Natl. Acad. Sci U.S.A 97, 8961-8966
69. McNemar, C., Snow, M. E., Windsor, W. T., Prongay, A., Mui, P., Zhang, R., Durkin, J., Le, H. V., and Weber, P. C. (1997) Biochemistry 36, 10006-10014
70. Stites, W. E. (1997) Chem Rev 97, 1233-1250
71. Kaufman, S. L. (1995) J. Aerosol Sci. 29, 537-552
72. Knutson, E. O. and Whitby, K. T. (1975) J. Aerosol Sci. 6, 451
73. Provencher, S. W. (1976) Biophys. J 16, 27-41
74. Banerjee, Y., Mizuguchi, J., Iwanaga, S., and Kini, R. M. (2005) J. Biol. Chem. 280, 42601-42611
75. Loo, J. A., Berhane, B., Kaddis, C. S., Wooding, K. M., Xie, Y., Kaufman, S. L., and Chernushevich, I. V. (2005) J. Am. Soc. Mass Spectrom 16, 998-1008
76. Papish, A. L., Tari, L. W., and Vogel, H. J. (2002) Biophys. J 83, 1455-1464
77. Spolar, R. S. and Record, M. T., Jr. (1994) Science 263, 777-784
78. Zhang, Y. L., Yao, Z. J., Sarmiento, M., Wu, L., Burke, T. R., Jr., and Zhang, Z. Y. (2000) J. Biol. Chem. 275, 34205-34212
79. Baker, B. M. and Murphy, K. P. (1997) J. Mol. Biol. 268, 557-569
80. Guinto, E. R. and Di Cora, E. (1996) Biochemistry 35, 8800-8804
81. Wintrode, P. L. and Privalov, P. L. (1997) J. Mol. Biol. 266, 1050-1062
82. Doyle, M. L. and Hensley, P. (1998) Methods Enzymol. 295, 88-99
83. Ortiz-Salmeron, E., Nuccetelli, M., Oakley, A. J., Parker, M. W., Lo, B. M., and Garcia-Fuentes, L. (2003) J. Biol. Chem. 278, 46938-46948
84. Kini, R. M. (2002) Clin Exp Pharmacol. Physiol 29, 815-822
85. Wilkins, D. K., Grimshaw, S. B., Receveur, V., Dobson, C. M., Jones, J. A., and Smith, L. J. (1999) Biochemistry 38, 16424-16431
86. Lu, H., Isralewitz, B., Krammer, A., Vogel, V., and Schulten, K. (1998) Biophys. J 75, 662-671
87. Longhi, S., Receveur-Brechot, V., Karlin, D., Johansson, K., Darbon, H., Bhella, D., Yeo, R., Finet, S., and Canard, B. (2003) J. Biol. Chem 278, 18638-18648
88. Perozzo, R., Folkers, G., and Scapozza, L. (2004) J Recept. Signal. Transduct. Res. 24, 1-52
89. Weber, P. C. and Salemme, F. R. (2003) Curr. Opin. Struct. Biol. 13, 115-121
90. Velazquez-Campoy, A., Leavitt, S. A., and Freire, E. (2004) Methods Mol. Biol. 261, 35-54
91. Dunitz, J. D. (1995) Chem. Biol. 2, 709-712
92. Cooper, A. (1999) Curr. Opin. Chem. Biol. 3, 557-563
93. Lumry, R. (2003) Biophys. Chem. 105, 545-557
94. Prabhu, N. V. and Sharp, K. A. (2005) Annu. Rev Phys. Chem. 56, 521-548
95. Murphy, K. P. and Freire, E. (1992) Adv. Protein Chem 43, 313-361
96. Livingstone, J. R., Spolar, R. S., and Record, M. T., Jr. (1991) Biochemistry 30, 4237-4244
97. Nicholls, A., Sharp, K. A., and Honig, B. (1991) Proteins 11, 281-296
98. Matulis, D. and Bloomfield, V. A. (2001) Biophys. Chem 93, 53-65
99. Sundberg, E. J., Urrutia, M., Braden, B. C., Isern, J., Tsuchiya, D., Fields, B. A., Malchiodi, E. L., Tormo, J., Schwarz, F. P., and Mariuzza, R. A. (2000) Biochemistry 39, 15375-15387
100. Tanford, C. (1978) Science 200, 1012-1018
101. Hibbits, K. A., Gill, D. S., and Willson, R. C. (1994) Biochemistry 33, 3584-3590
102. Tello, D., Goldbaum, F. A., Mariuzza, R. A., Ysern, X., Schwarz, F. P., and Poljak, R. J. (1993) Biochem. Soc. Trans. 21, 943-946
103. Schwarz, F. P., Tello, D., Goldbaum, F. A., Mariuzza, R. A., and Poljak, R. J. (1995) Eur. J Biochem. 228, 388-394
104. Tsumoto, K., Ueda, Y., Maenaka, K., Watanabe, K., Ogasahara, K., Yutani, K., and Kumagai, I. (1994) J Biol. Chem 269, 28777-28782
105. Tsumoto, K., Ogasahara, K., Ueda, Y., Watanabe, K., Yutani, K., and Kumagai, I. (1995) J Biol. Chem 270, 18551-18557
106. Faiman, G. A. and Horovitz, A. (1997) J Biol. Chem. 272, 31407-31411
107. Weber-Bornhauser, S., Eggenberger, J., Jelesarov, I., Bernard, A., Berger, C., and Bosshard, H. R. (1998) Biochemistry 37, 13011-13020
108. Keown, M. B., Henry, A. J., Ghirlando, R., Sutton, B. J., and Gould, H. J. (1998) Biochemistry 37, 8863-8869
109. Cole, J. L. and Garsky, V. M. (2001) Biochemistry 40, 5633-5641
110. Katragadda, M., Morikis, D., and Lambris, J. D. (2004) J Biol. Chem 279, 54987-54995
111. Ladbury, J. E., Wright, J. G., Sturtevant, J. M., and Sigler, P. B. (1994) J Mol. Biol. 238, 669-681
112. Morton, C. J. and Ladbury, J. E. (1996) Protein Sci 5, 2115-2118
113. Bhat, T. N., Bentley, G. A., Boulot, G., Greene, M. I., Tello, D., Dall'Acqua, W., Souchon, H., Schwarz, F. P., Mariuzza, R. A., and Poljak, R. J. (1994) Proc. Natl. Acad. Sci U.S.A 91, 1089-1093
114. Holdgate, G. A., Tunnicliffe, A., Ward, W. H., Weston, S. A., Rosenbrock, G., Barth, P. T., Taylor, I. W., Pauptit, R. A., and Timms, D. (1997) Biochemistry 36, 9663-9673
115. Sevrioukova, I. F., Li, H., Zhang, H., Peterson, J. A., and Poulos, T. L. (1999) Proc. Natl. Acad. Sci U.S.A 96, 1863-1868
116. Kornblatt, J. A., Kornblatt, M. J., Hoa, G. H., and Mauk, A. G. (1993) Biophys. J 65, 1059-1065
117. Xavier, K. A., Shick, K. A., Smith-Gil, S. J., and Willson, R. C. (1997) Biophys. J 73, 2116-2125

1. A polypeptide that comprises the amino acid sequence set forth in SEQ ID NO.1 or SEQ ID NO.3 or a variant, mutant or fragment thereof.

2. A polypeptide that comprises the amino acid sequence set forth in SEQ ID NO.2, 4 or 5 or a variant, mutant or fragment thereof.

3. A polypeptide according to claim 1 or 2, wherein said polypeptide is obtained from the venom of Hemachatus haemachatus (African Ringhals cobra).

4. A polypeptide according to claim 1, wherein said polypeptide exhibits anticoagulant activity.

5. A polypeptide comprising a functional equivalent of a polypeptide according to any one of claims 1 to 4, wherein said functional equivalent retains the activity of a polypeptide selected from the group consisting of SEQ. ID NO.1, SEQ. ID NO.2, SEQ. ID NO.3, SEQ. ID NO.4 and SEQ. ID NO.5.

6. A nucleic acid molecule which:
(i) encodes a polypeptide according to any one of claims 1 to 5; or
(ii) hybridizes to a nucleic acid molecule of part (i) or a variant, mutant, fragment or complement thereof.

7. The oligonucleotide according to claim 6, wherein said oligonucleotide is a primer or a probe.

8. A vector containing a nucleic acid molecule according to claim 6.

9. A host cell transformed with a vector according to claim 8.

10. A method of producing a polypeptide according to any one of claims 1 to 5, the method comprising culturing a host cell according to claim 9 under conditions suitable for the expression of the polypeptide according to any one of claims 1 to 5.

11. A method of producing a polypeptide according to any one of claims 1 to 5, the method comprising the chemical synthesis of said polypeptide.

12. The method of claim 11, wherein the chemical synthesis is solid-phase peptide synthesis.

13. The method according to any one of claims 10 to 12, wherein the method further comprises the step of purifying said polypeptide.

14. A method of generating a complex comprising:
(i) a polypeptide according to claim 1; and
(ii) a polypeptide according to claim 2,
wherein the method comprises contacting a polypeptide according to claim 1 with a polypeptide according to claim 2 under conditions suitable to allow formation of the complex.

15. A complex comprising:
(i) a polypeptide according to claim 1; and
(ii) a polypeptide according to claim 2.

16. The complex according to claim 15, wherein the ratio of (i) to (ii) is in the range of 1:2 to 2:1.

17. A method of generating an antibody which recognizes a polypeptide according to any one of claims 1 to 5 or a complex according to claim 15, wherein the method comprises the steps of:
(i) immunizing an animal with a polypeptide according to any one of claims 1 to 5 or a complex according to claim 15; and
(ii) obtaining the antibody from said animal.

18. An antibody which recognizes a polypeptide according to any one of claims 1 to 5 or a complex according to claim 15.

19. A method of producing an antivenom against a polypeptide according to any one of claims 1 to 5 or a complex according to claim 15, wherein the method comprises immunizing an animal with a polypeptide according to any one of claims 1 to 5 or a complex according to claim 15 and harvesting antibodies from the animal for use in the production of an antivenom.

20. An antivenom effective against a polypeptide according to any one of claims 1 to 5 or a complex according to claim 15.

21. A method for identifying a modulator of a polypeptide according to any one of claims 1 to 5 or a complex according to claim 15, the method comprising the steps of:
(i) contacting a test compound with a polypeptide according to any one of claims 1 to 5 or a complex according to claim 15; and
(ii) determining if the test compound binds to said polypeptide or said complex.

22. The method according to claim 21 further comprising the step of determining if the test compound increases or decreases the activity of said polypeptide or said complex.

23. A pharmaceutical composition comprising a polypeptide according to any one of claims 1 to 5, a nucleic acid molecule according to claim 6, a vector according to claim 8, a host cell according to claim 9, a complex according to claim 15, an antibody according to claim 18, an antivenom according to claim 20 or a modulator identified by the method according to claim 21.

24. A polypeptide according to any one of claims 1 to 5, a nucleic acid molecule according to claim 6, a vector according to claim 8, a host cell according to claim 9, a complex according to claim 15, an antibody according to claim 18, an antivenom according to claim 20 or a modulator identified by the method according to claim 21 for use in medicine.

25. A combined preparation for use in medicine, the combined preparation comprising:
(i) a polypeptide according to claim 1 or a nucleic acid molecule encoding the same; and
(ii) a polypeptide according to claim 2 or a nucleic acid molecule encoding the same.

26. The combined preparation of claim 25, wherein said combined preparation is for treating a patient in need of anticoagulant therapy.

27. Use of a polypeptide according to any one of claims 1 to 5, a nucleic acid molecule according to claim 6, a vector according to claim 8, a host cell according to claim 9 or a complex according to claim 15 in the manufacture of a medicament for use in treating a patient in need of anticoagulant therapy.

28. Use of:
(i) a polypeptide according to claim 1 or a nucleic acid molecule encoding the same; and
(ii) a polypeptide according to claim 2 or a nucleic acid molecule encoding the same in the manufacture of a combined preparation for treating a patient in need of anticoagulant therapy.

29. A method of treating a patient in need of anticoagulant therapy, the method comprising administering to the patient a polypeptide according to any one of claims 1 to 5, a nucleic acid molecule according to claim 6, a vector according to claim 8, a host cell according to claim 9, a complex according to claim 15 or a pharmaceutical composition according to claim 23.

30. A method of treating a patient in need of anticoagulant therapy, the method comprising administering to the patient:
(i) a polypeptide according to claim 1 or a nucleic acid molecule encoding the same; and
(ii) a polypeptide according to claim 2 or a nucleic acid molecule encoding the same.

31. A method of treating snake-bite in a patient, the method comprising administering to the patient a polypeptide according to any one of claims 1 to 5, a nucleic acid molecule according to claim 6, a vector according to claim 8, a host cell according to claim 9, a complex according to claim 15 or a pharmaceutical composition according to claim 23.

32. Use of a polypeptide according to any one of claims 1 to 5, a nucleic acid molecule according to claim 6, a vector according to claim 8, a host cell according to claim 9, a complex according to claim 15 or a pharmaceutical composition according to claim 23 in the manufacture of a medicament for treating snake-bite in a patient.
